(19) United States
(12) Patent Application Publication
(10) Pub. No.: US 2010/0092948 A1
(43) Pub. Date: Apr. 15, 2010

(54) BIOLOGICAL BAR CODE

(75) Inventors: James Davis, Carlsbad, CA (US); Mitchell D. Eggers, La Costa, CA (US); Rafael Ibarra, San Diego, CA (US); John Sadler, Belmont, CA (US); David Wong, Escondido, CA (US)

Correspondence Address: COOLEY GODWARD KRONISH LLP;ATTN: Patent Group, Suite 1100, 777 - 6th Street, NW, WASHINGTON, DC, 20001, US

(73) Assignee: GENVAULT CORPORATION, Carlsbad, CA (US)

(21) Appl. No.: 12/176,307

(22) Filed: Jul. 18, 2008

Related U.S. Application Data
(63) Continuation of application No. 10/836,119, filed on Apr. 29, 2004.
Application No. 10/836,119 is a continuation-in-part of application No. 10/426,940, filed on Apr. 29, 2003, now abandoned.

(51) Int. Cl. C12Q 1/68; G01N 33/48

(52) U.S. Cl. 435/6; 436/94

(57) ABSTRACT

The invention provides compositions and methods useful for identifying, verifying or authenticating any type of sample, whether the sample, is biological or non-biological.

### RELATED APPLICATIONS

This application is a divisional of co-pending U.S. Non-Provisional application Ser. No. 10/836,119, filed on Apr. 29, 2004, which is a Continuation-in-part of U.S. application Ser. No. 10/426,940, now abandoned, filed Apr. 29, 2003. The contents of the above-named applications are incorporated herein by reference.

### DESCRIPTION OF THE TEXT FILE SUBMITTED ELECTRONICALLY

The contents of the text file submitted electronically herewith are incorporated herein by reference in their entirety: A computer readable format copy of the Sequence Listing of the Sequence Listing (filename: GENV-007/03US Seqlist_ST25.txt, date recorded: Oct. 21, 2008, file size 10.6 kilobytes).

### TECHNICAL FIELD

The invention relates to compositions and methods of identifying samples to ensure their validity, authenticity or accuracy, and more particularly to bar-coded samples and archives, methods of bar-coding samples, and methods of identifying, validating, and authenticating bar-coded samples in which the coding may be done with biological molecules, modified forms or derivatives thereof.

### BACKGROUND

Identification of anonymized DNA samples from human patients can be difficult if the samples are in liquid form and are subject to error during handling. Many other biological and non-biological samples can be confused or subject to identification error. Barcode labels on tubes or containers offer only partial solution of the identification problem as they can fall off, be obscured, removed or otherwise made unreadable. Furthermore, such barcode labels are easily counterfeited. A nucleic acid sample offers a built in identification code but is only useful if the identity information for that nucleic acid is at hand or can be obtained. Long, unique, oligonucleotide sequences have been added to samples as a means of identification but this requires that a unique sequence be synthesized for each and every sample and costly sequencing analysis to identify the oligonucleotide sequences. The invention addresses the inadequacies of present identification methods and provides related advantages.

### SUMMARY

The invention provides compositions allowing identification of a sample, samples uniquely identified by the compositions and methods of producing identified samples and identifying samples so produced. For example, a composition of the invention including two or more oligonucleotides can be added to a sample, in which each of the oligonucleotides do not specifically hybridize to the sample, in which each of the oligonucleotides are physically or chemically different from each other (e.g., their length or sequence), and are in a unique combination that allows identification of the sample.

In one embodiment, a composition includes two or more oligonucleotides and a sample, the oligonucleotides denoted a first oligonucleotide set, the first oligonucleotide set comprising oligonucleotides incapable of specifically hybridizing to said sample, the oligonucleotides having a length from about 8 nucleotides to 50 Kb. The first oligonucleotide set includes oligonucleotides each having a physical or chemical difference from the other oligonucleotides of the first oligonucleotide set, and, optionally the first oligonucleotide set includes one or more oligonucleotides having a different sequence therein capable of specifically hybridizing to a unique primer pair denoted a first primer set. In one aspect, the difference is oligonucleotide length. In various additional aspects, the set includes two oligonucleotides denoted A through B and the unique combination comprises A with or without B; or B with or without A; the set includes three oligonucleotides denoted A through C and the unique combination comprises A with or without B or C; B with or without A or C; or C with or without A or B; the set includes four oligonucleotides denoted A through D and the unique combination comprises A with or without B or C or D; B with or without A or C or D; C with or without A or B or D; or D with or without A or B or C; the set includes five oligonucleotides denoted A through E and the unique combination comprises A with or without B or C or D or E; B with or without A or C or D or E; C with or without A or B or D or E; D with or without A or B or C or E; or E with or without A or B or C or D; the set includes six oligonucleotides denoted A through F and the unique combination comprises A with or without B or C or D or E or F; B with or without A or C or D or E or F; C with or without A or B or D or E or F; D with or without A or B or C or E or F; E with or without A or B or C or D or F; or F with or without A or B or C or D or E; or the set includes seven oligonucleotides denoted A through G and the unique combination comprises A with or without B or C or D or E or F or G; B with or without A or C or D or E or F or G; C with or without A or B or D or E or F or G; D with or without A or B or C or E or F or G; E with or without A or B or C or D or F or G; F with or without A or B or C or D or E or G; or G with or without A or B or C or D or E or F.

In additional embodiments, a unique combination includes two to five, five to ten, 10 to 15, 15 to 20, 20 to 25, 25 to 30, 30 to 40, 40 to 50, 50 to 75, 75 to 100, or more oligonucleotides. Oligonucleotides within a set can have the same or a different sequence length, e.g., differ by at least one nucleotide. In one aspect, the oligonucleotides have a length from about 10 to 5000 base pairs; 10 to 3000 base pairs; 12 to 1000 base pairs; 12 to 500 base pairs; 15 to 250 base pairs; or 18 to 250, 20 to 200, 20 to 150, 25 to 150, 25 to 100, or 25 to 75 base pairs. Oligonucleotides can be single, double or triple strand deoxyribonucleic acid (DNA) or ribonucleic acid (RNA).

In an additional embodiment, a composition includes two or more oligonucleotides and a sample, the two or more oligonucleotides of two or more oligonucleotide sets. In one aspect, a composition therefore includes one or more oligonucleotides denoted a second oligonucleotide set, the second oligonucleotide set including oligonucleotides incapable of specifically hybridizing to the sample, the second oligonucleotide set comprising oligonucleotides having a length from about 8 nucleotides to 50 Kb. The second oligonucleotide set includes oligonucleotides each having a physical or chemical difference from the other oligonucleotides of the second oligonucleotide set, and optionally the second oligonucleotide set includes one or more oligonucleotides having a different sequence therein capable of specifically hybridizing to a unique primer pair denoted a second primer set. In additional aspects, one or more oligonucleotides from additional sets are added to the sample and the one or more oligonucleotides of the first and second oligonucleotide sets, e.g., one or more oligonucleotides denoted a third oligonucleotide set, the third oligonucleotide set including oligonucleotides incapable of specifically hybridizing to the sample, the third oligonucleotide set including oligonucleotides having a length from about 8 nucleotides to 50 Kb, the third oligonucleotide set including oligonucleotides each having a physical or chemical difference from the other oligonucleotides of the third oligonucleotide set and optionally the third oligonucleotide set includes one or more oligonucleotides having a different sequence therein capable of specifically hybridizing to a unique primer pair denoted a third primer set; one or more oligonucleotides denoted a fourth oligonucleotide set, the fourth oligonucleotide set including oligonucleotides incapable of specifically hybridizing to the sample, the fourth oligonucleotide set including oligonucleotides having a length from about 8 nucleotides to 50 Kb, the fourth oligonucleotide set including oligonucleotides each having a physical or chemical difference from the other oligonucleotides of the fourth oligonucleotide set, and optionally the fourth oligonucleotide set includes one or more oligonucleotides having a different sequence therein capable of specifically hybridizing to a unique primer pair denoted a fourth primer set; one or more oligonucleotides denoted a fifth oligonucleotide set, the fifth oligonucleotide set including oligonucleotides incapable of specifically hybridizing to the sample, the fifth oligonucleotide set including oligonucleotides having a length from about 8 nucleotides to 50 Kb, the fifth oligonucleotide set including oligonucleotides each having a physical or chemical difference from the other oligonucleotides of the fifth oligonucleotide set, and optionally the fifth oligonucleotide set includes one or more oligonucleotides having a different sequence therein capable of specifically hybridizing to a unique primer pair denoted a fifth primer set; one or more oligonucleotides denoted a sixth oligonucleotide set, the sixth oligonucleotide set including oligonucleotides incapable of specifically hybridizing to the sample, the sixth oligonucleotide set including oligonucleotides having a length from about 8 nucleotides to 50 Kb, the sixth oligonucleotide set including oligonucleotides each having a physical or chemical difference from the other oligonucleotides of the sixth oligonucleotide set and optionally the sixth oligonucleotide set includes one or more oligonucleotides having a different sequence therein capable of specifically hybridizing to a unique primer pair denoted a sixth primer set; and so on and so forth. In a particular aspect, the difference is in oligonucleotide length. In additional aspects, the one or more oligonucleotides of the first, second, third, fourth, fifth, sixth, etc., oligonucleotide set has the same or a different length as an oligonucleotide of the first, second, third, fourth, fifth, sixth, etc., oligonucleotide set. In further aspects, the one or more oligonucleotides of each additional oligonucleotide set, e.g., third, fourth, fifth, sixth, etc., has the same or a different length as an oligonucleotide of the first, second, third, fourth, etc. oligonucleotide set. Thus, for example, in one aspect, an oligonucleotide of the first, second, third, fourth, fifth or sixth oligonucleotide set has the same or a different length as an oligonucleotide of the second, third, fourth or fifth oligonucleotide set, respectively.

In yet additional embodiments, a composition includes one or more unique primer pairs of a primer set, e.g., a composition that includes oligonucleotides denoted a first, second, third, fourth, fifth, sixth, etc., set includes a first primer set that specifically hybridizes to one or more of the oligonucleotides denoted the first set. In still further embodiments, a composition that includes oligonucleotides denoted a first, second, third, fourth, fifth, or sixth, etc., set includes a first, second, third, fourth, fifth, or sixth, etc. primer set that specifically hybridizes to one or more of the oligonucleotides denoted the first, second, third, fourth, fifth, or sixth, etc. set. The primers of the unique primer pairs can have any length, e.g., a length from about 8 to 250, 10 to 200, 10 to 150, 10 to 125, 12 to 100, 12 to 75, 15 to 60, 15 to 50, 18 to 50, 20 to 40, 25 to 40 or 25 to 35 nucleotides. The primers of the unique primer pairs can have a length of about 9/10, ⅘, ¾, 7/10, ⅗, ½, ⅖, ⅓, 3/10, ¼, ⅕, ⅙, 1/7, ⅛, 1/10 of the length of the oligonucleotide to which the primer binds. Primers can bind at or near the 3′ or 5′ terminus of the oligonucleotide, e.g., within about 1 to 25 nucleotides of the 3′ or 5′ terminus of the oligonucleotide. Primers can have the same or different lengths, e.g., each primer of the unique primer pair differs in length from about 0 to 50, 0 to 25, 0 to 10, or 0 to 5 base pairs; can be entirely or partially complementary to all or at least a part of one or more of the oligonucleotides, e.g., 40-60%, 60-80%, 80-95% or more (primers need not be 100% homologous or have 100% complementarity); and can be 100% complementary to a sequence.

Samples include any physical entity. Exemplary samples include pharmaceuticals, biologicals and non-biological samples. Non-biological samples include any document (e.g., evidentiary document, a testamentary document, an identification card, a birth certificate, a signature card, a driver's license, a social security card, a green card, a passport, a letter, or a credit or debit card), currency; bond, stock certificate, contract, label, piece of art, recording medium (e.g., digital recording medium), electronic device, mechanical or musical instrument, precious stone or metal, or dangerous device (e.g., firearm, ammunition, an explosive or a composition suitable for preparing an explosive).

Biological samples include foods (meats or vegetables such as beef, pork, lamb, fowl or fish), beverages (alcohol or non-alcohol). Biological samples include tissue samples, forensic samples, and fluids such as blood, plasma, serum, sputum, semen, urine, mucus, cerebrospinal fluid and stool. Biological samples further include any living or non-living cell, such as an egg or sperm, bacteria or virus, pathogen, nucleic acid (mammalian such as human or non-mammalian), protein, carbohydrate. Typically, a sample that is nucleic acid will have less than 50% homology with the different sequence of the oligonucleotides or the primer pairs, such that the oligonucleotides or primer pairs do not specifically hybridize to the nucleic acid to the extent that it prevents developing the code. Thus, in particular aspects, for a nucleic acid that is bacterial the oligonucleotides do not specifically hybridize to the bacterial nucleic acid, for a nucleic acid that is viral the oligonucleotides do not specifically hybridize to the viral nucleic acid.

Oligonucleotides can be modified, e.g., to be nuclease resistant. Compositions can include preservatives, e.g., nuclease inhibitors such as EDTA, EGTA, guanidine thiocyanate or uric acid. Oligonucleotides can be mixed with, added to or imbedded within the sample, e.g., attached to, applied to, affixed to or imbedded within a substrate (permeable, semi-permeable or impermeable two dimensional surface or three dimensional structure, e g., a plurality of wells). Oligonucleotides can be physically separable or inseparable from the substrate, e.g., under conditions where the sample remains substantially attached to the substrate the oligonucleotides can be separated.

In yet further embodiments, a composition includes three or more unique primer pairs and two or more oligonucleotides, optionally in combination with a sample, wherein the unique primer pairs are denoted a first, second, third, fourth, fifth, or sixth, etc. primer set, each of the unique primer pairs having a different sequence, at least two of the unique primer pairs capable of specifically hybridizing to two oligonucleotides, wherein the oligonucleotides are denoted a first, second, third, fourth, fifth, or sixth, etc. oligonucleotide set, the oligonucleotides having a length from about 8 nucleotides to 50 Kb. The oligonucleotides in each set have a physical or chemical difference from the other oligonucleotides comprising the same oligonucleotide set. In various aspects, a composition includes additional unique primer pairs, e.g., four or more unique primer pairs, five or more unique primer pairs, six or more unique primer pairs. In additional aspects, a composition includes additional oligonucleotides, e.g., three, four, five, six or more oligonucleotides, etc. In still further aspects, a composition includes one or more oligonucleotides denoted a second, third, fourth, fifth, sixth, etc. oligonucleotide set, the oligonucleotide(s) of the second, third, fourth, fifth, sixth, etc. oligonucleotide set including one or more oligonucleotides having a different sequence therein capable of specifically hybridizing to a unique corresponding primer pair denoted a second, third, fourth, fifth, sixth, etc. primer set, the second, third, fourth, fifth, sixth, etc. oligonucleotide set including oligonucleotides incapable of specifically hybridizing to the sample, the second, third, fourth, fifth, sixth, etc. oligonucleotide set including oligonucleotides having a length from about 8 nucleotides to 50 Kb, the second, third, fourth, fifth, sixth, etc. oligonucleotide set including oligonucleotides each having a physical or chemical difference from the other oligonucleotides comprising the second, third, fourth, fifth, sixth, etc. oligonucleotide set.

In still additional embodiments, a composition of the invention is in an organic or aqueous solution having one or more phases (compatible with polymerase chain reaction (PCR)), slurry, semi-solid, or a solid. In further embodiments, a composition of the invention is included within a kit.

The invention also provides methods of producing bio-tagged samples. In one embodiment, a method includes selecting a combination of two or more oligonucleotides to add to a sample, the oligonucleotides, optionally from two or more oligonucleotide sets, incapable of specifically hybridizing to the sample, the oligonucleotides having a length from about 8 to 5000 nucleotides, and the oligonucleotides within each set having a physical or chemical difference (e.g., oligonucleotide length or sequence), and adding the combination of two or more oligonucleotides to the sample, wherein the combination of oligonucleotides identifies the sample, thereby producing a bio-tagged sample. In one aspect, one or more of the oligonucleotides has a different sequence therein capable of specifically hybridizing to a unique primer pair.

The invention further provides methods of identifying bio-tagged samples. In one embodiment, a method includes detecting in a sample the presence or absence of two or more oligonucleotides, wherein the oligonucleotides are identified based upon a physical or chemical difference, thereby identifying a combination of oligonucleotides in the sample; comparing the combination of oligonucleotides with a database including particular oligonucleotide combinations known to identify particular samples; and identifying the sample based upon which of the particular oligonucleotide combinations in the database is identical to the combination of oligonucleotides in the sample. In one aspect, sample identification is based upon the different lengths of the oligonucleotides. In another aspect, sample identification is based upon the different sequence of the oligonucleotides. In yet another aspect, identification does not require sequencing all of the oligonucleotides, e.g., identification is based upon a primer or primer pairs that specifically hybridizes to one or more of the oligonucleotides that identifies the sample. In still another aspect, identification is based upon the different lengths of the oligonucleotides, or by hybridization to two or more unique primer pairs having a different sequence, optionally followed by amplification (e.g., PCR).

The invention moreover provides archives of bio-tagged samples. In one embodiment, an archive includes a sample; and two or more oligonucleotides. The oligonucleotides are incapable of specifically hybridizing to the sample, the oligonucleotides have a length from about 8 to 50 Kb nucleotides, the oligonucleotides each have a physical or chemical difference (e.g., a different length or sequence), and optionally one or more of the oligonucleotides have a different sequence therein capable of specifically hybridizing to a unique primer pair, the oligonucleotides are in a unique combination that identifies the sample; and a storage medium for storing the bio-tagged samples.

The invention still further provides methods of producing archives of bio-tagged samples. In one embodiment, a method includes selecting a combination of two or more oligonucleotides to add to a sample, the oligonucleotides are incapable of specifically hybridizing to the sample, the oligonucleotides have a length from about 8 to 50 Kb nucleotides, the oligonucleotides each have a physical or chemical difference (e.g., a different length or sequence), one or more of the oligonucleotides have a different sequence therein capable of specifically hybridizing to a unique primer pair; adding the combination of two or more oligonucleotides to the sample and placing the bio-tagged sample in a storage medium for storing the bio-tagged samples. The combination of oligonucleotides identifies the sample.

Substrates and arrays can further include one or more oligonucleotides, each capable of specifically hybridizing to one or more code oligonucleotides. In one embodiment, a substrate includes a plurality of polynucleotide or polypeptide sequences each immobilized at pre-determined positions, wherein at least two of the polypeptide or polynucleotide sequences are designated as target sequences and are distinct from each other, and a polynucleotide sequence designated as an identifier oligonucleotide that does not specifically hybridize to a nucleic acid that is capable of specifically hybridizing to the target sequences. In another embodiment, a substrate includes a plurality of polynucleotide sequences each immobilized at pre-determined positions on the substrate, wherein at least two polynucleotide sequences designated as target sequences are distinct from each other, and wherein at least a third polynucleotide sequence designated as an identifier oligonucleotide does not specifically hybridize to a nucleic acid that is capable of specifically hybridizing to the target sequences.

Methods of producing substrates and arrays, as well as methods of identifying the bio-tag or code of the sample (developing the code) with substrates and arrays, are also provided. In one embodiment, a method includes selecting a combination of two or more oligonucleotides to add to a substrate, the oligonucleotides, designated as identifier oligonucleotides each capable of specifically hybridizing to a code oligonucleotide; and adding the two or more identifier oligonucleotides to the substrate in a number sufficient to specifically hybridize to all oligonucleotides potentially present in a coded sample. In another embodiment, a method includes providing a substrate including two or more identifier oligonucleotides, wherein the number of identifier oligonucleotides are sufficient to specifically hybridize to all code oligonucleotides potentially present in a coded sample; contacting the substrate with a coded sample; and detecting specific hybridization between the identifier oligonucleotides and code oligonucleotides present in the sample, thereby identifying the code oligonucleotides present in the sample. Comparing the combination of code oligonucleotides with a database including particular oligonucleotide combinations known to identify particular samples identifies the code and, therefore, the sample, based upon the particular oligonucleotide combination in the database that is identical to the oligonucleotide code of the sample.

Methods of producing archives of substrates and arrays capable of identifying a sample code are further provided. In one embodiment, a method includes selecting two or more identifier oligonucleotides to add to a substrate, each identifier oligonucleotide capable of specifically hybridizing to a corresponding code oligonucleotide; adding the two or more identifier oligonucleotides to the substrate, wherein the number of identifier oligonucleotides are sufficient to specifically hybridize to all oligonucleotides potentially present in a coded sample; and placing the substrate or array in a storage medium.

Computer systems, media and instructions for producing or selecting a bio-tag (code), identifying a bio-tag (code), applying a bio-tag (code) to a sample are further provided. In one embodiment, a computer readable medium encoded with data and instructions for producing a bio-tag for identifying a sample causes an apparatus executing the instructions to: identify a bio-tag code for the sample; associate a unique combination of oligonucleotides with the bio-tag code, wherein the unique combination of oligonucleotides identifies the sample; provide the unique combination of oligonucleotides to a predetermined location on a sample carrier; and create a data record associating the unique combination of oligonucleotides with the predetermined location. In another embodiment, a computer readable medium encoded with data and instructions for applying a bio-tag to a sample carrier cause an apparatus executing the instructions to: retrieve a container containing a selected bio-tag; the bio-tag comprising a unique combination of oligonucleotides; confirm that the selected bio-tag is available for use; provide the bio-tag to a predetermined location on a sample carrier; and create a data record associating the bio-tag with the predetermined location. In yet another embodiment, a computer executed method of producing a bio-tag for identifying a sample includes: identifying a bio-tag code for the sample; associating a unique combination of oligonucleotides with the bio-tag code; and creating a data record associating the unique combination of oligonucleotides with a predetermined location on a sample carrier. In still another embodiment, a computer executed method of identifying a bio-tagged sample includes: detecting specific hybridization between a code oligonucleotide and a respective (corresponding) identifier oligonucleotide maintained at a predetermined location on a substrate; identifying one or more code oligonucleotides that are present in the bio-tagged sample in accordance with the detecting; comparing the code oligonucleotides present in the bio-tagged sample to data records associating unique oligonucleotide combinations with unique samples; and identifying the bio-tagged sample responsive to the comparing.

### DESCRIPTION OF DRAWINGS

FIGS. 1A and 1B illustrate exemplary codes, A) 534523151, or in binary form, 10100 01000 10010 00101 10001; and B) 530523151, or in binary form, 10100 00000 10010 00101 10001, following size-based fractionation of amplified oligonucleotides. Lanes are as follows: 1, a ladder of 5 oligonucleotides with lengths of 60, 70, 80, 90, and 100 nucleotides; 2, primer set #1 amplified oligonucleotides; 3, primer set #2 amplified oligonucleotides; 4, primer set #3 amplified oligonucleotides; 5, primer set #4 amplified oligonucleotides; 6, primer set #5 amplified oligonucleotides. Sets 1-5 are multiplex primer sets for each of the 5 oligonucleotide sets.

FIG. 2A is a simplified diagram illustrating a code generated following size-based fractionation via gel electrophoresis and indicating an alternative convention for reading the code.

FIG. 2B is a simplified diagram illustrating the binary code read in accordance with the convention indicated in FIG. 2B.

FIG. 3A is a simplified diagram illustrating one embodiment of a sample carrier.

FIG. 3B is a simplified diagram illustrating an exemplary code associated with one bio-tag maintained at different locations on the sample carrier of FIG. 3A.

FIG. 4 is a simplified flow diagram illustrating the general operation of one embodiment of a method of producing a bio-tag for use in identifying a sample.

FIG. 5 is a simplified flow diagram illustrating the general operation of one embodiment of a method of applying a bio-tag to a sample carrier.

FIG. 6 is photograph of an agarose gel showing size-based separation of coding oligonucleotides following PCR amplification, as described in Example 1 for 50, 75, and 100 by coding oligonucleotides.

FIG. 7 is a photograph of an agarose gel showing size-based separation of coding oligonucleotides following PCR amplification, as described in Example 1 for 50, 60, 70, 80, 90, and 100 by coding oligonucleotides.

FIG. 8 is a photograph of an agarose gel showing size-based separation of coding oligonucleotides following PCR amplification, as described in Example 1 for 50, 75, and 100 by coding oligonucleotides. The template used in the different lanes of FIG. 8 included no template (control), FTA™ paper containing human blood either with or without coding oligonucleotides, and IsoCode™ page containing human blood either with or without coding oligonucleotides.

FIG. 9 is a photograph of a polyacrylamide gel showing size-based separation of coding oligonucleotides following PCR amplification, as described in Example 2 for 50, 60, 70, 80, 90, and 100 by coding oligonucleotides from Set #2.

FIG. 10 is a photograph of a polyacrylamide gel showing size-based separation of coding oligonucleotides following PCR amplification, as described in Example 2 for 50, 60, 70, 80, 90, and 100 by coding oligonucleotides from Set #3.

FIG. 11 is a photograph of an agarose gel showing size-based separation of b-actin sequences PCR amplified from blood samples that had been applied to matices, as described in Example 3.

### DETAILED DESCRIPTION

The invention is based at least in part on compositions including oligonucleotides that are physically or chemically different from each other (e.g., in their length and/or sequence), and that are in a unique combination. Adding to or mixing a unique combination of oligonucleotides with a given sample, i.e., coding the sample, allows the sample to be identified based upon the combination of oligonucleotides added or mixed. By determining the oligonucleotide combination (the “code” or “bio-tag”) in a query sample and comparing the oligonucleotide combination to oligonucleotide combinations known to identify particular samples (e.g., a database of known oligonucleotide combinations that identify samples), the query sample is thereby identified. Thus, where it is desired to identify, verify or authenticate a sample, a unique combination of oligonucleotides can be added to or mixed with the sample (to “code” or “tag” the sample), and the sample can subsequently be identified, verified or authenticated based upon the particular unique combination of oligonucleotides present in the sample.

As a non-limiting illustration of the invention, from a pool of 25 oligonucleotides, each oligonucleotide having a different sequence in order to avoid specific hybridization with other oligonucleotides, and each oligonucleotide having a different length (in this example, five lengths: 60, 70, 80, 90 and 100 nucleotides), nine are added to a sample. The nine oligonucleotides added to the sample (the “code”) are recorded and the code optionally stored in a database. The oligonucleotide code is developed using primer pairs that specifically hybridize to each oligonucleotide that is present. In this particular illustration, there are 25 oligonucleotides possible and 5 sets of primer pairs (denoted primer Sets 1-5). Each set of primer pairs specifically hybridize to 5 oligonucleotides and, therefore, by using 5 primer sets, all 25 oligonucleotides potentially present in the sample are identified. In this illustration, the nine oligonucleotides present in the sample which specifically hybridize to a corresponding primer pair are identified by polymerase chain reaction (PCR) based amplification. In contrast, because the other 16 oligonucleotides are absent from the sample these oligonucleotides will not be amplified by the primers that specifically hybridize to them. Thus, differential primer hybridization among the different oligonucleotides is used to identify which oligonucleotides, among those possibly present, that are actually present in the sample.

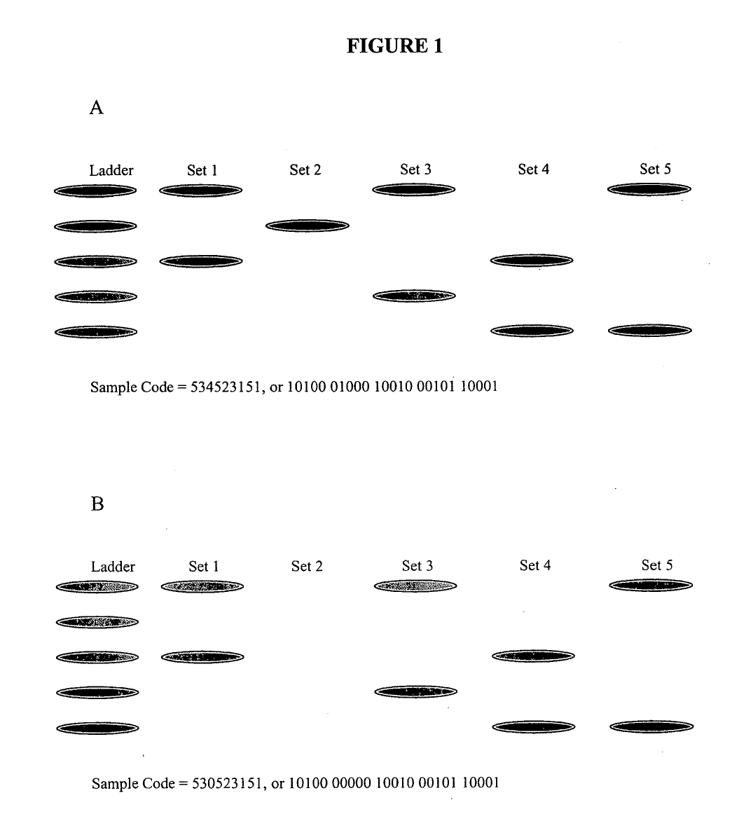

Following PCR, the 5 reactions containing amplified products, which in this illustration reflect both the oligonucleotide length and the sequence of the region that hybridizes to the primers, are size-fractionated via gel electrophoresis: each reaction representing one primer set is fractionated in a single lane for a total of 5 lanes (Sets 1-5, which correspond to FIG. 1, lanes 2-6, respectively). The developed “bar-code” in this illustration is the pattern of the fractionated amplified products in each lane. In this illustration, the 60, 70, 80, 90 and 100 base oligonucleotides correspond to code numbers 1, 2, 3, 4 and 5, respectively, and the bar code is read beginning with lane 2, from top to bottom, and each lane thereafter, 534523151 (FIG. 1A). Alternatively, the bar-code may be designated as a binary number, where each of the 25 possible oligonucleotides at the 60, 70, 80, 90 and 100 positions in all 5 lanes is designated by a “1” or a “0” based upon the presence or absence, respectively, of the oligonucleotide (amplified product) at that particular position. Thus, in FIG. IA the corresponding binary number would read 10100 01000 10010 00101 10001.

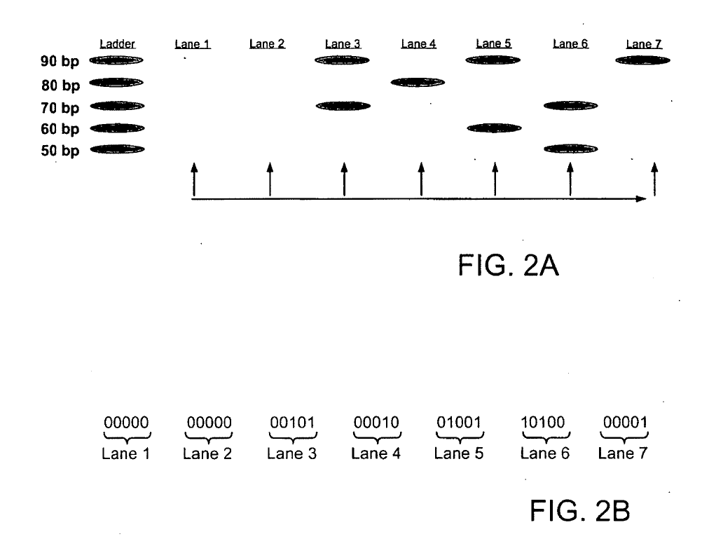

In the exemplary illustration (FIGS. 1 and 2) each primer set amplifies at least one oligonucleotide. However, because not all oligonucleotides need be present, oligonucleotides ‘for a given primer set may be completely absent. That is, a code where an oligonucleotide is absent is designated by a “0.” Thus, for example, where there is no oligonucleotide present that specifically hybridizes to a primer pair in primer set #2, the code would read: 530523151 (FIG. 1B), and the corresponding binary number for lane 2 would be “0” at each position, which would read 10100 00000 10010 00101 10001.

In order to develop the “code” in the exemplary illustration (FIGS. 1 and 2), every primer pair that specifically hybridizes to every oligonucleotide from the pool of 25 oligonucleotides is used in the amplification reactions. The initial screen for which oligonucleotides are actually present in the sample is therefore based upon differential primer hybridization and subsequent amplification of the oligonucleotide(s) that hybridizes to a corresponding primer pair. Thus, every one of the 25 oligonucleotides potentially present in the sample can be identified because all primer pairs that specifically hybridizes to all oligonucleotides are used in the screen. In the illustration, five primer sets are used, each primer set containing 5 primer pairs. Five separate reactions were performed with the 5 primer pairs in each primer set to amplify all 25 oligonucleotides. Thus, although primer pair may be present in any given reaction, if the oligonucleotide that specifically hybridizes to the primer pair is absent from that reaction, the oligonucleotide will not be amplified.

Following the reactions, the oligonucleotides (amplified products) are differentiated from each other based upon differences in their length. Thus, in the context of developing the code, oligonucleotides comprising the code need not be subject to sequencing analysis in order to identify or distinguish them from one another. Accordingly, the invention does not require that the oligonucleotides comprising the code be sequenced in order to develop the code.

In the exemplary illustration (FIGS. 1 and 2), the “code” is developed by dividing the sample containing the oligonucleotides into five reactions and separately amplifying the reactions with each primer set. For example, a coded sample that is applied or attached to a substrate (e.g., a small 3 mm diameter matrix) can be divided into 5 pieces and the amplification reactions performed on each of the 5 pieces of substrate, each reaction having a different primer set. Optionally, the oligonucleotides could first be eluted from the substrate and the eluent divided into five separate reactions. As an alternative approach to separate reactions, the substrate can be subjected to 5 sequential reactions with each primer set. For example, if the oligonucleotide code is applied or attached to a substrate the code can be developed by performing 5 sequential amplification reactions on the substrate, and removing the amplified products after each reaction before proceeding to the next reaction. The amplified products from each of the 5 sequential reactions are then fractionated separately to develop the code.

If desired fewer oligonucleotides can be used, optionally in a single dimension. A set of oligonucleotides or amplified products can be fractionated in a single dimension, e.g., one lane. For example, where a large number of unique codes is not anticipated to be needed 2, 3, 4, 5, 6, 7, 8, 9, 10, etc. oligonucleotides can be a code in a single lane format. A corresponding single primer set would therefore include 2, 3, 4, 5, 6, 7, 8, 9, 10, etc. numbers of unique primer pairs in order to detect/identify the 2, 3, 4, 5, 6, 7, 8, 9, 10, oligonucleotides, respectively, that may be present. Given sufficient resolving power of the separation system, essentially there is no upper limit to the number of oligonucleotides that can be separated in one dimension. Thus, there may be 10-15, 15-20, 20-25, 25-30, 30-35, 35-40, 40-45, 45-50, etc., or more oligonucleotides that may be separated in a single dimension. Accordingly, invention compositions can contain unlimited numbers of oligonucleotides in one or more oligonucleotide sets. A given primer set therefore also need not be limited; the number of primer pairs in a primer set will reflect the number of oligonucleotides desired to be amplified, e.g., 10-15, 15-20, 20-25, 25-30, 30-35, 35-40, 40-45, 45-50, etc., or more oligonucleotides.

Thus, in one embodiment the invention provides compositions including two or more oligonucleotides and a sample; the oligonucleotides denoted a first oligonucleotide set, the first oligonucleotide set including oligonucleotides incapable of specifically hybridizing to the sample, the first oligonucleotide set oligonucleotides having a length from about 8 to 50 Kb nucleotides, the first oligonucleotide set oligonucleotides each having a physical or chemical difference (e.g., a different length) from the other oligonucleotides comprising the first oligonucleotide set, and the first oligonucleotide set oligonucleotides each having a different sequence therein capable of specifically hybridizing to a unique primer pair denoted a first primer set. In one aspect, the first oligonucleotide set oligonucleotides are in a unique combination allowing identification of the sample. In additional aspects, the two oligonucleotides are denoted A and B, and the composition includes A with or without B, or B alone; the three oligonucleotides are denoted A through C and the composition includes A with or without B or C, B with or without A or C, or C with or without A or B; the four oligonucleotides are denoted A through D and the composition includes A with or without B or C or D, B with or without A or C or D, C with or without A or B or D, or D with or without A or B or C; the five oligonucleotides are denoted A through E and the compositions includes A with or without B or C or D or E, B with or without A or C or D or E, C with or without A or B or D or E, D with or without A or B or C or E, or E with or without A or B or C or D; the six oligonucleotides are denoted A through F and the composition includes A with or without B or C or D or E or F, B with or without A or C or D or E or F, C with or without A or B or D or E or F, D with or without A or B or C or E or F, E with or without A or B or C or D or F, or F with or without A or B or C or D or E; the seven oligonucleotides are denoted A through G and the composition includes A with or without B or C or D or E or F or G, B with or without A or C or D or E or F or G, C with or without A or B or D or E or F or G, D with or without A or B or C or E or F or G, E with or without A or B or C or D or F or G, F with or without A or B or C or D or E or G, or G with or without A or B or C or D or E or F. In yet further aspects, the first oligonucleotide set includes a unique combination of two to five, five to ten, 10 to 15, 15 to 20, 20 to 25, 25 to 30, 30 to 40, 40 to 50, 50 to 100, or more oligonucleotides.

As used herein, the term “physical or chemical difference,” and grammatical variations thereof, when used in reference to oligonucleotide(s), means that the oligonucleotide(s) has a physical or chemical characteristic that allows one or more of the oligonucleotides to be distinguished from each another. In other words, the oligonucleotides have a difference that allows them to be distinguished from one or more other oligonucleotides and, therefore, identified when present among the other oligonucleotides. One particular example of a physical difference is oligonucleotide length. Another particular example of a physical difference is oligonucleotide sequence. Additional examples of physical differences that allow oligonucleotides to be distinguished from each other, which may in part be influenced by oligonucleotide length or sequence, include charge, solubility, diffusion rate, and absorption. Examples of chemical differences include modifications as set forth herein, such as molecular beacons, radioisotopes, fluorescent moieties, and other labels. As discussed, when developing the code sequencing of the oligonucleotides is not required.

Generally, as used herein for convenience purposes the oligonucleotide sets are designated according to the primer sets used to amplify them. Thus, in the exemplary illustration (FIGS. 1 and 2), primer set #1 amplifies oligonucleotide set #1; primer set #2 amplifies oligonucleotide set #2; primer set #3 amplifies oligonucleotide set #3; primer set #4 amplifies oligonucleotide set #4; primer set #5 amplifies oligonucleotide set #5; primer set #6 amplifies oligonucleotide set #6; primer set #7 amplifies oligonucleotide set #7; primer set #8 amplifies oligonucleotide set #8, primer set #9 amplifies oligonucleotide set #9; primer set #10 amplifies oligonucleotide set #10, etc.

In the above exemplary illustration, primer set #1 amplified products (oligonucleotides) are size-fractionated in lane 2, primer set #2 amplified products (oligonucleotides) are size-fractionated in lane 3, primer set #3 amplified products (oligonucleotides) are size-fractionated in lane 4, primer set #4 amplified products (oligonucleotides) are size-fractionated in lane 5, and primer set #5 amplified products (oligonucleotides) are size-fractionated in lane 6 (FIG. 1). However, amplified products need not be fractionated in any particular lane in order to obtain the correct code, provided that the primers used to produce the amplified products are known and the reactions are separately fractionated. That is, by knowing which primers are used in the amplification reaction, e.g., primer set #1 specifically hybridizes to and amplifies oligonucleotides of set #1, the amplified products and, therefore, the oligonucleotides detectable are also known. Thus, amplified products can be fractionated in any order (lane) since the primers that specifically hybridize to particular oligonucleotides are known. For example, if the correct code is obtained by reading the amplified products from primer sets #1-#5 in order, but the primer sets are fractionated out of order, (e.g., primer set #1 is run in lane 2 and primer set #2 is run in lane 1) the code can be corrected by merely reading lane 2 (primer set #1) before lane 1 (primer set #2). Accordingly, amplified products can be fractionated in any order to develop the code because they can be “read” to correspond with the order of the primer set that provides the correct code.

In the exemplary illustration (FIGS. 1 and 2), oligonucleotides amplified with primer sets #1-5 are separately size fractionated in 5 lanes to develop the code (FIG. 1, five lanes, beginning with primer set #1 in lane 2). Even though an invention code can be employed in which oligonucleotides are fractionated in a single lane following amplification with one primer set, using multiple primer sets and fractionating oligonucleotides in multiple lanes provides a more convenient format and expands the number of unique codes available within that format in comparison to fractionating in a single dimension (one lane). The number of different code combinations can be represented as 2^{n(m)}, where “n” represents the number of oligonucleotides per lane and “m” represents the number of lanes. Thus, in this exemplary illustration, 25 oligonucleotides in a 5×5 format (5 oligonucleotides per lane in 5 lanes) provides 2²⁵ different code combinations, or 33,554,432 codes. In contrast, 5 oligonucleotides in a 5×1 format (5 oligonucleotides in one lane) provides 2⁵ different code combinations, or 32 codes

In the exemplary illustration (FIGS. 1 and 2) the amplified products fractionated in a single lane (one set of oligonucleotides corresponding to one primer set) are physically or chemically different from each other (e.g., have a different length, charge, solubility, diffusion rate, adsorption, or label) in order to be distinguished from each other. Thus, in addition to increasing the number of available codes, an advantage of fractionating in multiple lanes is that the oligonucleotides or amplified products fractionated in different lanes can have one or more identical physical or chemical characteristics yet still be distinguished from each other. For example, using two dimensions allows oligonucleotides in different sets to have the same length since each set is separately fractionated from the other set(s) (e.g., each set is fractionated in a different lane). Furthermore, each oligonucleotide can have the same sequence. As the number of oligonucleotides fractionated in a given lane increase, a broader size range for the oligonucleotides in order to fractionate them and, consequently, greater resolving power of the fractionation system may be needed in order to develop the code. Thus, where length is used to distinguish between the oligonucleotides within a given set, because the oligonucleotides in different sets can have identical lengths, the oligonucleotides used for the code can have a narrower size range and be fractionated with comparatively less resolving power. The use of multiple dimensions for size fractionation is also more convenient than one dimension since fewer primers are present in a given reaction mix.

Thus, in accordance with the invention there are provided compositions including multiple oligonucleotide sets and a sample. In one embodiment, oligonucleotides denoted a first oligonucleotide set include oligonucleotides incapable of specifically hybridizing to the sample, the oligonucleotides having a length from about 8 to 50 Kb nucleotides, oligonucleotides each having a physical or chemical difference (e.g., a different length) from the other oligonucleotides comprising the first oligonucleotide set, the oligonucleotides each having a different sequence therein capable of specifically hybridizing to a unique primer pair denoted a first primer set; and oligonucleotides denoted a second oligonucleotide set include oligonucleotides each having a different sequence therein capable of specifically hybridizing to a unique primer pair denoted a second primer set, incapable of specifically hybridizing to the sample, a length from about 8 to 50 Kb nucleotides, and each have a physical or chemical difference (e.g., a different length) from the other oligonucleotides comprising said second oligonucleotide set.

In another embodiment, compositions include two oligonucleotide sets and a third oligonucleotide set, the third oligonucleotide set including oligonucleotides each having a different sequence therein capable of specifically hybridizing to a unique primer pair denoted a third primer set, incapable of specifically hybridizing to the sample, a length from about 8 to 50 Kb nucleotides, and each having a physical or chemical difference (e.g., a different length) from the other oligonucleotides of the third oligonucleotide set.

In a further embodiment, compositions include three oligonucleotide sets and a fourth oligonucleotide set, the fourth oligonucleotide set including oligonucleotides each having a different sequence therein capable of specifically hybridizing to a unique primer pair denoted a fourth primer set, incapable of specifically hybridizing to the sample, a length from about 8 to 50 Kb nucleotides, and each having physical or chemical difference (e.g., a different length) from the other oligonucleotides of the fourth oligonucleotide set.

In an additional embodiment, compositions include four oligonucleotide sets and a fifth oligonucleotide set, the fifth oligonucleotide set including oligonucleotides each having a different sequence therein capable of specifically hybridizing to a unique primer pair denoted a fifth primer set, incapable of specifically hybridizing to the sample, a length from about 8 to 50 Kb nucleotides, and each having a physical or chemical difference (e.g., a different length) from the other oligonucleotides of the fifth oligonucleotide set. In various aspects of the invention, in the compositions including multiple oligonucleotide sets, one or more oligonucleotides of the second, third, fourth, fifth, sixth, etc., oligonucleotide set has a physical or chemical characteristic that is the same as one or more oligonucleotides of any other oligonucleotide set (e.g., an identical nucleotide length).

The number of oligonucleotides that may be selected from for producing a coded sample may initially be large enough to account for potentially large numbers of samples or be increased as the number of samples coded increases. For example, where there are few samples to be coded, in one dimension (one lane), 2 unique oligonucleotides provide 4 unique codes (2²), e.g., in binary form, 00, 01, 10, 11; for 3 unique oligonucleotides 8 unique codes are available (2³), e.g., in binary form, 000, 001, 010, 100, 011, 110, 101, 111; for 4 unique oligonucleotides 16 unique codes are available (2⁴); for 5 unique oligonucleotides 32 unique codes are available (2⁵). To expand the number of available codes, one need only increase the number of different oligonucleotides. For example, for 6 unique oligonucleotides 64 unique codes are available (2⁶); for 7 unique oligonucleotides 128 unique codes are available (2⁷); for 8 there are 256 codes available; for 9 there are 512 codes available; for 10 there are 1,024 codes available; for 11 there are 2,048 codes available; for 12 there are 4,096 codes available; for 13 there are 8,192 codes available; for 14 there are 16,384 codes available; for 15 there are 32,768 codes available; for 16 there are 65,536 codes available; for 17 there are 131,072 codes available; for 18 there are 262,144 codes available; for 19 there are 524,288 codes available; for 20 there are 1,048,576 codes available; for 21 there are 2,097,152 codes available; for 22 there are 4,194,304 codes available; for 23 there are 8,388,608 codes available; for 24 there are 16,777,216 codes available; for 25 there are 33,554,432 codes available; etc. Thus, where the number of samples exceeds the available codes, where there are an unknown number of samples to be coded, or where it is desired that the number of codes available be in excess of the projected number samples, additional different oligonucleotides may be added to the oligonucleotide pool from which the oligonucleotides are selected for the code, or the coding may employ an initial large number of different oligonucleotides in order to provide an unlimited number of unique oligonucleotide combinations and, therefore, unique codes. For example, 30 different oligonucleotides provides over one billion unique codes (1,073,741,824 to be precise).

A third dimension could be added in order to expand the code. Adding a third dimension would expand the number of codes available to 2^{(m)np}, where “p” represents the third dimension. Thus, adding a third dimension to a 5×5 format as in the exemplary illustration (FIGS. 1 and 2), 2^{25(p)} different unique codes are available. One example of a third dimension could be based upon isoelectric point or molecular weight. For example, a unique peptide tag could be added to one or more of the oligonucleotides and the code fractionated using isoelectric focusing or molecular weight alone, or in combination, e.g., 2D gel electrophoresis.

The code can include additional information. For example, a code can include a check code. By using the number of oligonucleotides in each lane a check can be embedded with the code. For example, in FIG. IA, lanes 2-6 have 2, 1, 2, 2 and 2 oligonucleotides, respectively. The check code in this case would be 21222. For FIG. 1B, the check code would be 20222.

The code output can be “hashed,” if desired, so that the code loses any characteristics that would allow it to be traced back to the original sample or the patient that provided the sample. For example, each number in 534523151 could be increased or decreased by one, 645634262 and 423412040, respectively.

The term “hybridization,” “annealing” and grammatical variations thereof refers to the binding between complementary nucleic acid sequences. The term “specific hybridization,” when used in reference to an oligonucleotide capable of forming a non-covalent bond with another sequence (e.g., a primer), or when used in reference to a primer capable of forming a non-covalent bond with another sequence (e.g., an oligonucleotide) means that the hybridization is selective between 1) the oligonucleotide and 2) the primer. In other words, the primer and oligonucleotide preferentially hybridize to each other over other nucleic acid sequences that may be present (e.g., other oligonucleotides, primers, a sample that is nucleic acid, etc.) to the extent that the oligonucleotides present can be identified to develop the code.

Suitable positive and negative controls, for example, target and non-target oligonucleotides or other nucleic acid can be tested for amplification with a particular primer pair to ensure that the primer pair is specific for the target oligonucleotide. Thus, the target oligonucleotide, if present, is amplified by the primer pair whereas the non-target oligonucleotides, non-target primers or other nucleic acid are not amplified to the extent they interfere with developing the code. False negatives, i.e., where an oligonucleotide of the code is present but not detected following amplification, can be detected by correlating the oligonucleotides of the code that are detected with the various codes that are possible. For example, a gel scan of the correct code(s) can be provided to the end user in order to allow the user to match the code detected with one of the gel scan codes. Where the end user is dealing with a limited number of codes, even if one or a few oligonucleotides are not detected, the correct code can readily be identified by matching the detected code with the gel scan of the possible codes that may be available, particularly where the number of available codes possible is large. More particularly for example, an end user requests 10 coded samples from an archive for sample analysis. The coded samples are retrieved from the archive and forwarded to the end user who subsequently analyzes the samples. In order to ensure that a particular sample subsequently analyzed corresponds to the sample received from the archive, the end user then wishes to determine the code for that sample. However, one of the oligonucleotides of the code in that sample is not detected during the analysis of the code, producing an incomplete code. Because the codes for all samples forwarded to the end user are known, the incomplete code can be fully completed based on the code to which the incomplete code most closely corresponds. Alternatively, all codes received by the end user could be developed and, by a process of elimination the incomplete code is developed.

For two nucleic acid sequences to hybridize, the temperature of a hybridization reaction must be less than the calculated TM (melting temperature). As is understood by those skilled in the art, the TM refers to the temperature at which binding between complementary sequences is no longer stable. The TM is influenced by the amount of sequence complementarity, length, composition (% GC), type of nucleic acid (RNA vs. DNA), and the amount of salt, detergent and other components in the reaction. For example, longer hybridizing sequences are stable at higher temperatures. Duplex stability between RNAs or DNAs is generally in the order of RNA:RNA>RNA:DNA>DNA:DNA. All of these factors are considered in establishing appropriate conditions to achieve specific hybridization (see, e.g., the hybridization techniques and formula for calculating TM described in Sambrook et al., 1989, supra). Generally, stringent conditions are selected to be about 5° C. lower than the melting point (Tm) for the specific sequence at a defined ionic strength and pH.

Exemplary conditions used for specific hybridization and subsequent amplification for developing the exemplary code (FIGS. 1 and 2) are disclosed in Example 1. One exemplary condition for PCR is as follows: Buffer (1×): 16 mM (NH₄)₂SO₄, 67 mM Tris-HCl (pH 8.8 at 25° C.), 0.01% Tween 20, 1.5 mM MgCl₂; dNTP: 200 uM each; primer concentration: 62.5 mM of each primer (all 5 primer pairs present in each reaction); enzyme: 2 units of Biolase (Taq; Bioline, Randolph, Mass.); PCR cycling conditions: 93° C. for 2 minutes, 55° C. for 1 minute, 72° C. for 2 minutes, followed by 29 cycles of 93° C. for 30 seconds, 55° C. for 30 seconds, 72° C. for 45 seconds. Conditions that vary from the exemplary conditions include, for example, primer concentrations from about 20 mM to 100 mM; enzyme from about 1 unit to 4 units; PCR Cycling conditions, annealing temperatures from about 49° C. -59° C., and denaturing, annealing, and elongation time from about 30 seconds-2 minutes. Of course, the skilled artisan recognizes that the conditions will depend upon a number of factors including, for example, the number of oligonucleotides and primers used, their length and the extent of complementarity. Those skilled in the art can determine appropriate conditions in view of the extensive knowledge in the art regarding the factors that affect PCR (see, e.g., Molecular Cloning: A Laboratory Manual 3^{rd} ed., Joseph Sambrook, et al., Cold Spring Harbor Laboratory Press; (2001); Short Protocols in Molecular Biology 4^{th} ed., Frederick M. Ausubel (ed.), et al., John Wiley & Sons; (1999); and Pcr (Basics: From Background to Bench) 1^{st} ed., M. J. McPherson et al., Springer Verlag (2000)).

As used herein, the term “incapable of specifically hybridizing to a sample” and grammatical variants thereof, when used in reference to an oligonucleotide or a primer, means that the oligonucleotide or primer does not specifically hybridize to the sample (e.g., a nucleic acid sample) to the extent that any non-specific hybridization occurring between one or more oligonucleotides or primers and the nucleic acid sample does not interfere with developing the code. Thus, for example where a sample is human nucleic acid, typically all or a part of the oligonucleotide sequence will be non-human (e.g., bacterial, viral, yeast, etc.) such that any non-specific hybridization occurring between one or more oligonucleotides or primers and the human nucleic acid does not interfere with oligonucleotide detection/identification, i.e., identifying the code.

There may be situations where an oligonucleotide or a primer specifically hybridizes to a sample and some amplification of the sample may occur thereby producing a false positive. However, rarely if ever will the size of the false product be the expected size of an oligonucleotide that is a part of the code. Furthermore, a threshold level can be set such that the amount of an oligonucleotide must be greater than that threshold in order for the oligonucleotide to be considered “present” or “positive.” If the amount of the oligonucleotide or amplified product produced is greater than the threshold level then the product is considered present. In contrast, if the amount is less than the threshold level, then the oligonucleotide or amplified product is considered a false positive. Visual inspection of relative amounts or other quantification means using densitometers or gel scanners can be used to determine whether or not a given product is above or below a certain threshold.

Accordingly, oligonucleotide(s) and primer(s) that specifically hybridize to each other can be entirely non-complementary to a sample that is nucleic acid, or have some or 100% complementarity, provided that any hybridization occurring between the oligonucleotide(s) or primer(s) and the nucleic acid sample does not interfere with developing the code. It is therefore intended that the meaning of “incapable of specifically hybridizing to a sample” used herein includes situations where an oligonucleotide or a primer specifically hybridizes to a sample and amplification of the sample may occur, but the amplification does not interfere with developing the code. “Incapable of specifically hybridizing” also can be used to refer to the absence of specific hybridization among the different oligonucleotides used to code or tag the sample, among primer pairs used for amplification, and between primers and non-target oligonucleotides, to the extent that even if some hybridization occurs, the hybridization does not prevent the code from being developed.

In addition, when there is nucleic acid present in the sample that is ancillary to the sample, that is, for a protein sample or any other non-nucleic acid sample in which nucleic acid happens to be present but is not the sample that is coded, an oligonucleotide or primer may also specifically hybridize to the nucleic acid provided that the hybridization with the nucleic acid sample does not interfere with developing the code. Because the size of any amplified product produced will not have the expected size of the oligonucleotide, such hybridization will rarely if ever interfere with developing the code. Furthermore, in a situation where there is nucleic acid ancillary to the sample, typically the amount of primer(s) is in excess of the nucleic acid such that no interference with developing the code occurs.

Thus, in particular embodiments of the invention, the oligonucleotide(s) or primer(s) will have less than about 40-50% homology with a sample that is nucleic acid. In additional specific embodiments, the oligonucleotide(s) will have less that about 0.5-50% homology, e.g., 45%, 40%, 35%, 30%, 25%, 20%, 15%, 10%, 5%, 3%, or less homology with a sample that is nucleic acid.

The oligonucleotides used for coding the sample may be of any length. For example, oligonucleotides can range in length from 8-10 nucleotides to about 100 Kb in length. In specific embodiments, the oligonucleotides have a length from about 10 nucleotides to about 50 Kb, from about 10 nucleotides to about 25 Kb, from about 10 nucleotides to about 10 Kb, from about 10 nucleotides to about 5 Kb; from about 12 nucleotides to about 1000 nucleotides, from about 15 nucleotides to about 500 nucleotides, from about 20 nucleotides to 250 nucleotides, or from about 25 to 250 nucleotides, 30 to 250 nucleotides, 35 to 200 nucleotides, 40 to 150 nucleotides, 40 to 100 nucleotides, or 50 to 90 nucleotides.

Where the physical difference used for oligonucleotide identification is length, the length differs by at least one nucleotide. Typically, oligonucleotides will differ in sequence length from each other, for example, by 1 to 500, 1 to 300, 1 to 200, 3 to 200, 5 to 150, 5 to 120, 5 to 100, 5 to 75, or 5 to 50 nucleotides; or 2-5, 5-10, 10-20, 20-30, 30-50, 50-100, 100-250, 250-500 or more nucleotides. More typically, the length difference can be in a range convenient for size-fractionation via gel-electrophoresis, for example, 5, 10, 15, 20, 25, 30, 35, 40, 45, 50 nucleotide lengths are convenient to detect differences in the size of oligonucleotides having a length a range from about 20 to 5000 nucleotides.

In the exemplary illustration (FIGS. 1 and 2), the oligonucleotides are amplified and subsequently fractionated via gel electrophoresis. The code however may be developed by any other means capable of differentiating between the oligonucleotides comprising the code. For example, the oligonucleotides whether amplified or not may be fractionated by size-exclusion, paper or ion-exchange chromatography, or be separated on the basis of charge, solubility, diffusion or adsorption. Thus, the means of identifying the oligonucleotides of the code include any method which differentiates between oligonucleotides that may be present in the code.

For example, oligonucleotides having a chemical or physical difference that cannot be differentiated by size-fractionation or differential hybridization may be differentiated by other means including modifying the oligonucleotides. As set forth in detail below, oligonucleotides may be labeled using any of a variety of detectable moieties in order to differentiate them from each other. As such, a code may include one or more oligonucleotides that have an identical nucleotide sequence or length but that have some other chemical or physical difference between them that allows them to be distinguished from each other. Accordingly, such oligonucleotides, which may be included in a code as set forth herein, need not be subject to hybridization or subsequent amplification in order to determine their presence and consequently, the code identity.

As used herein, the term “different sequence,” when used in reference to oligonucleotides, means that the nucleotide sequences of the oligonucleotides are different from each other to the extent that the oligonucleotides can be differentiated from each other. The different sequence of an oligonucleotide “capable of specifically hybridizing to a unique primer pair” or an identifier oligonucleotide “capable of specifically hybridizing to a unique oligonucleotide of a code” therefore includes any contiguous sequence that is suitable for primer or identifier oligonucleotide hybridization such that the code oligonucleotide can be differentiated on the basis of differential hybridization from other oligonucleotides potentially present. The oligonucleotides will differ in sequence from each other by at least one nucleotide, but typically will exhibit greater differences to minimize non-specific hybridization, e.g., 2-5, 5-10, 10-20, 20-30, 30-50, 50-100, 100-250, 250-500 or more nucleotides in the oligonucleotides will differ from the other oligonucleotides. The number of nucleotide differences to achieve differential hybridization and, therefore, oligonucleotide differentiation will be influenced by the size of the oligonucleotide, the sequence of the oligonucleotide, the assay conditions (e.g., hybridization conditions such as temperature and the buffer composition), etc. Oligonucleotide sequence differences may also be expressed as a percentage of the total length of the oligonucleotide sequence, e.g., when comparing the two oligonucleotides, the percentage of the nucleotides that are either identical or different from each other. Thus, for example, for a 30 by oligonucleotide (OL1) as little as 20-25% of the sequence need be different from another oligonucleotide sequence (OL2) in order to differentiate between OL1 and OL2, provided that the sequences of OL1 and OL2 that are 75-80% identical do not interfere with developing the code.

The term “different sequence,” when used in reference to oligonucleotides, refers to oligonucleotides in which differential hybridization is used to differentiate among the oligonucleotides comprising the code. This does not preclude the presence of other oligonucleotides in the code where differential primer hybridization is not used to identify them. For example, two or more oligonucleotides of the code can have an identical nucleotide sequence where a primer pair hybridizes. Thus, such oligonucleotides are not distinguished from each other on the basis of length or differential primer hybridization. However, oligonucleotides having the same primer hybridization sequence can have different sequence length, or some other physical or chemical difference such as charge, solubility, diffusion adsorption or a label, such that they can be differentiated from each other. For example, code oligonucleotides having shared primer hybridization sites can be differentiated from each other due to the presence of a different sequence outside of the primer hybridization sites, either a sequence region that flanks a primer binding site or a sequence region that is located between the primer binding sites. Specific hybridization between such a “non-primer binding site” sequence region and a complementary identifier oligonucleotide identifies the particular code oligonucleotide. Accordingly, oligonucleotides of the code can have the same nucleotide sequence where a primer pair hybridizes and as such, a primer pair can specifically hybridize to two or more oligonucleotides of the code.

The oligonucleotide sequence determines the sequence of the primer pairs or identifier oligonucleotides used to detect the oligonucleotides. As disclosed herein, using unique primer pairs or identifier oligonucleotides that specifically hybridize to each of the oligonucleotides potentially present in a query sample facilitates detection of all oligonucleotides. Typically, the corresponding primer pairs hybridize to a portion of the oligonucleotide sequence. Thus, the sequence region to which the primers or identifier oligonucleotides hybridize is the only nucleotide sequence that need be known in order to detect the oligonucleotide. In other words, in order to detect or identify any oligonucleotide of the code, only the nucleotide sequence that participates in hybridization needs to be known. Accordingly, nucleotide sequences of an oligonucleotide that do not participate in specific hybridization with a primer pair or identifier oligonucleotide can be any sequence or unknown.

For example, where the primer pairs hybridize at the 5′ or 3′ end of an oligonucleotide, the intervening sequence between the hybridization sites can be any sequence or can be unknown. Likewise, for primer pairs that hybridize near the 5′ or 3′ end of an oligonucleotide, the intervening sequence between the primer hybridization sites or the sequences that flank the primer hybridization sites can be any sequence or can be unknown. Likewise, for identifier oligonucleotides, the portion that does not hybridize to its corresponding complementary code oligonucleotide can be any sequence or can be unknown. In either case, nucleotides located between or that flank the hybridization sites can be any sequence or unknown, provided that the intervening or flanking sequences do not hybridize to different oligonucleotides, non-target identifier oligonucleotides, non-target primers or to a sample that is nucleic acid to such an extent that it interferes with developing the code.

Since the nucleotide sequence of the oligonucleotides to which the primers or identifier oligonucleotides hybridize confer hybridization specificity which in turn indicates the identity of the oligonucleotide (e.g., OL1), nucleotides that do not participate in hybridization may be identical to nucleotides in different oligonucleotides (e.g., OL2) that do not participate in hybridization. For example, if a particular oligonucleotide is 30 nucleotides in length (OL1), a primer or identifier oligonucleotide could be as few as 8 nucleotides meaning that 14 nucleotides in the oligonucleotide are not participating in hybridization. Thus, all or a part of these 14 contiguous nucleotides in OL1 can be identical to one or more of the other oligonucleotides in the same set or in a different set (e.g., OL2, OL3, OL4, OL5, OL6, etc.), provided that the primer pairs or identifier oligonucleotides that specifically hybridize to OL2, OL3, OL4, OL5, OL6, etc., do not also hybridize to this 14 nucleotide sequence to the extent that this interferes with developing the code. Accordingly, nucleotide sequences regions within an oligonucleotide that do not participate in hybridization may be identical to other oligonucleotides, in part or entirely.

The location of the different sequence capable of specifically hybridizing to a unique primer pair in an oligonucleotide will typically be at or near the 5′ and 3′ termini of the oligonucleotide. The location of the different sequence capable of specifically hybridizing to a unique primer pair in the oligonucleotide is influenced by oligonucleotide length. For example, for shorter oligonucleotides the location of the different sequence capable of specifically hybridizing to a unique primer pair is typically at or near the 5′ and 3′ termini. In contrast, with longer oligonucleotides the location of the different sequence capable of specifically hybridizing to a unique primer pair can be further away from the 5′ and 3′ termini. Where oligonucleotide size differences are used for identification, there need only be size differences between the oligonucleotides in the code or in the amplified oligonucleotide products. Thus, if the oligonucleotides are detected in the absence of amplification, the sizes of the oligonucleotides will be different from each other. In contrast, if amplification is used to develop the code as in the exemplary illustration (FIGS. 1 and 2), the primers in a given set need only specifically hybridize to the oligonucleotides in the set (i.e., not at the 5′ and 3′ termini) to produce amplified products having different sizes from each other. In other words, oligonucleotides within a given set can have an identical length provided that the primers specifically hybridize with the oligonucleotide at locations that produce amplified products having a different size. As an example, two oligonucleotides, OL1 and OL2, within a given set each have a length of 50 nucleotides. When developing the code primer pairs that specifically hybridize at the 5′ and 3′ termini of OL1 produce an amplified product of 50 nucleotides, whereas primer pairs that specifically hybridize 5 nucleotides within the 5′ and 3′ termini of OL2 produce an amplified product of 40 nucleotides.

Thus, the location of the different sequence capable of specifically hybridizing to a unique primer pair in an oligonucleotide can, but need not be, at the 5′ and 3′ termini of the oligonucleotide. In one embodiment, the different sequence is located within about 0 to 5, 5 to 10, 10 to 25 nucleotides of the 3′ or 5′ terminus of the oligonucleotide. In another embodiment, the different sequence is located within about 25 to 50 or 50 to 100 nucleotides of the 3′ or 5′ terminus of the oligonucleotide. In additional embodiments, the different sequence is located within about 100 to 250, 250 to 500, 500 to 1000, or 1000 to 5000 nucleotides of the 3′ or 5′ terminus of the oligonucleotide.

As used herein, the terms “oligonucleotide,” “nucleic acid,” “polynucleotide,” “primer,” and “gene” include linear oligomers of natural or modified monomers or linkages, including deoxyribonucleotides, ribonucleotides, and α-anomeric forms thereof capable of specifically hybridizing to a target sequence by way of a regular pattern of monomer-to-monomer interactions, such as Watson-Crick type of base pairing, base stacking, Hoogsteen or reverse Hoogsteen types of base pairing. Monomers are typically linked by phosphodiester bonds or analogs thereof to form the polynucleotides. Oligonucleotides can be a synthetic oligomer, a sense or antisense, circular or linear, single, double or triple strand DNA or RNA. Whenever an oligonucleotide is represented by a sequence of letters, such as “ATGCCTG,” the nucleotides are in a 5′ to 3′ orientation from left to right.

Essentially any polymer that has a unique sequence can be used for the code, provided the polymer is detectable and can be distinguished from other polymers present in the code. Polymers include organic polymers or alkyl chains identified by spectroscopy, e.g., NMR and FT-IR. Polymers include one or more amino acids attached thereto, for example, peptides derivatized with ninhydrin or opthaldehyde, which can be detected with a fluorometer. Polymers further include peptide nucleic acid (PNA), which refers to a nucleic acid mimic, e.g., DNA mimic, in which the deoxyribose phosphate backbone is replaced by a pseudopeptide backbone while retaining the natural nucleotides.

Oligonucleotides therefore include moieties which have all or a portion similar to naturally occurring oligonucleotides but which are non-naturally occurring. Thus, oligonucleotides may have one or more altered sugar moieties or inter-sugar linkages. Particular examples include phosphorothioate and other sulfur-containing species known in the art. One or more phosphodiester bonds of the oligonucleotide can be substituted with a structure that enhances stability of the oligonucleotide. Particular non-limiting examples of such substitutions include phosphorothioate bonds, phosphotriesters, methyl phosphonate bonds, short chain alkyl or cycloalkyl structures, short chain heteroatomic or heterocyclic structures and morpholino structures (U.S. Pat. No. 5,034,506). Additional linkages include those disclosed in U.S. Pat. Nos. 5,223,618 and 5,378,825.

Oligonucleotides therefore further include nucleotides that are naturally occurring, synthetic, and combinations thereof. Naturally occurring bases include adenine, guanine, cytosine, thymine, uracil and inosine. Particular non-limiting examples of synthetic bases include xanthine, hypoxanthine, 2-aminoadenine, 6-methyl, 2-propyl and other alkyl adenines, 5-halo uracil, 5-halo cytosine, 6-aza cytosine and 6-aza thymine, pseudo uracil, 4-thiuracil, 8-halo adenine, 8-aminoadenine, 8-thiol adenine, 8-thioalkyl adenines, 8-hydroxyl adenine and other 8-substituted adenines, 8-halo guanines, 8-amino guanine, 8-thiol guanine, 8-thioalkyl guanines, 8-hydroxyl guanine and other substituted guanines, other aza and deaza adenines, other aza and deaza guanines, 5-trifluoromethyl uracil, 5-trifluoro cytosine and tritylated bases.

Oligonucleotides can be made nuclease resistant during or following synthesis in order to preserve the code. Oligonucleotides can be modified at the base moiety, sugar moiety or phosphate backbone to improve stability, hybridization, or solubility of the molecule. For example, the 5′ end of the oligonucleotide may be rendered nuclease resistant by including one or more modified internucleotide linkages (see, e.g., U.S. Pat. No. 5,691,146).

The deoxyribose phosphate backbone of oligonucleotide(s) can be modified to generate Peptide nucleic acids (Hyrup et al., Bioorg. Med. Chem. 4:5 (1996)). The neutral backbone of PNAs allows specific hybridization to DNA and RNA under conditions of low ionic strength. The synthesis of PNA oligomers can be performed using standard solid phase peptide synthesis protocols (see, e.g., Perry-O'Keefe et al., Proc. Natl. Acad. Sci. USA 93:14670 (1996)). PNAs hybridize to complementary DNA and RNA sequences in a sequence-dependent manner, following Watson-Crick hydrogen bonding. PNA-DNA hybridization is more sensitive to base mismatches; PNA can maintain sequence discrimination up to the level of a single mismatch (Ray and Bengt, FASEB J. 14:1041 (2000)). Due to the higher sequence specificity of PNA hybridization, incorporation of a mismatch in the duplex considerably affects the thermal melting temperature. PNA can also be modified to include a label, and the labeled PNA included in the code or used as a primer or probe to detect the labeled PNA in the code. For example, a PNA light-up probe in which the asymmetric cyanine dye thiazole orange (TO) has been tethered. When the light-up PNA hybridizes to a target, the dye binds and becomes fluorescent (Svavnik et al., Analytical Biochem. 281:26 (2000)).

Compositions of the invention including oligonucleotides can include additional components or agents that increase stability or inhibit degradation of the oligonucleotides, i.e., a preservative. Particular non-limiting examples of preservatives include, for example, EDTA, EGTA, guanidine thiocyanate and uric acid.

As used herein, the term “unique primer pair” means a primer pair that specifically hybridizes to an oligonucleotide target under the conditions of the assay. As disclosed herein, a primer pair may hybridize to two or more oligonucleotides that are potentially present in the code. A unique primer pair need only be complementary to at least a portion of the target oligonucleotide such that the primers specifically hybridize and the code is developed. For example, oligonucleotide sequences from about 8 to 15 nucleotides are able to tolerate mismatches; the longer the sequence, the greater the number of mismatches that may be tolerated without affecting specific hybridization. Thus, an 8 to 15 base sequence can tolerate 1-3 mismatches; a 15 to 20 base sequence can tolerate 1-4 mismatches; a 20 to 25 base sequence can tolerate 1-5 mismatches; a 25 to 30 base sequence can tolerate 1-6 mismatches, and so forth.

As used herein, the term “identifier oligonucleotide” means an oligonucleotide that specifically hybridizes to a code oligonucleotide under the conditions of the assay. Specific hybridization between an identifier oligonucleotide and a code oligonucleotide identifies the code oligonucleotide as present, by producing a signal that indicates such hybridization. In contrast, identifier oligonucleotides that do not specifically hybridize to any code oligonucleotides do not produce a signal indicative of hybridization. As with unique primer pairs that specifically hybridize to code oligonucleotides, identifier oligonucleotides can have the same length, or be shorter or longer than the code oligonucleotides to which it specifically hybridizes. Additionally as with the unique primer pairs, identifier oligonucleotides need only be complementary to at least a portion of the target code oligonucleotide, such that the identifier oligonucleotide specifically hybridizes to code oligonucleotide and the code is developed. Of course, the longer the oligonucleotide sequence, the greater the number of nucleotide mismatches that may be tolerated without affecting specific hybridization between an identifier oligonucleotide and a complementary target code oligonucleotide.

The hybridization is specific in that the primer pair or identifier oligonucleotide does not significantly hybridize to non-target oligonucleotides or non-target identifier oligonucleotide, other primers or a sample that is nucleic acid to an extent that interferes with developing the code. Thus, primer pairs and identifier oligonucleotide can share partial complementary with non-target oligonucleotides because stringency of the hybridization or amplification conditions can be such that the primer pairs or identifier oligonucleotide preferentially hybridize to a target oligonucleotide(s). For example, in the case of a 30 base oligonucleotide, OL1, with 10 base primer pairs (Primers #1 and #2), and a 40 base oligonucleotide, OL2, with 10 base primer pairs (Primers #3 and #4), Primers #1 and #3 and/or Primers #2 and #4 can share sequence identity, for example, from 1 to about 5 contiguous nucleotides may be identical between Primers #1 and #3 and/or Primers #2 and #4 without interfering with developing the code. As length increases the number of contiguous nucleotides of a primer pair or identifier oligonucleotide that may be non-complementary with a target oligonucleotide increases. As length increases the number of contiguous nucleotides of a primer pair or identifier oligonucleotide that may be complementary with a non-target oligonucleotide or another primer likewise increases. Generally, the maximum number of contiguous nucleotides that may be identical between primers or identifier oligonucleotides targeted to different oligonucleotides without interfering with developing the code will be about 40-60%. In any event, the primers and identifier oligonucleotides need not be 100% homologous to or have 100% complementary with the target oligonucleotides.

Primer pairs and identifier oligonucleotides can be any length provided that they are capable of hybridizing to the target oligonucleotide and, where amplification is used to develop the code, capable of functioning for oligonucleotide amplification. In particular embodiments of the invention, one or more of the primers of the unique primer pairs has a length from about 8 to 250 nucleotides, e.g., a length from about 10 to 200, 10 to 150, 10 to 125, 12 to 100, 12 to 75, 15 to 60, 15 to 50, 18 to 50, 20 to 40, 25 to 40 or 25 to 35 nucleotides. In additional embodiments of the invention, one or more of the primers of the unique primer pairs has a length of about 9/10, ⅘, ¾, 7/10, ⅗, ½, ⅖, ⅓, 3/10, ¼, ⅕, ⅙, 1/7, ⅛, 1/10 of the length of the oligonucleotide to which the primer binds.

Individual primers in a primer pair, primer pairs in a primer set and primers of different sets can have the same or different lengths. In particular embodiments of the invention, each primer of a given unique primer pair, each primer pair in a primer set and primers in different primer sets have the same length or differ in length from about 1 to 500, 1 to 250, 1 to 100, 1 to 50, 1 to 25, 1 to 10, or 1 to 5 nucleotides.

In the exemplary illustration (FIGS. 1 and 2), the code is developed by specific hybridization to primers and subsequent amplification and size-fractionation of the oligonucleotides that hybridize to the primers via electrophoresis. In addition to alternative ways of size-fractionation of the oligonucleotides, which include, size-exclusion, ion-exchange, paper and affinity chromatography, diffusion, solubility, adsorption, there are alternative methods of code development. For example, oligonucleotides could be amplified, then subsequently cleaved with an enzyme to produce known fragments with known lengths that could be the basis for a code. Alternatively, if a sufficient amount of oligonucleotide is present, the oligonucleotides may be size-fractionated without hybridization and subsequent amplification and directly visualized (e.g., electrophoretic size fractionation followed by UV fluorescence). Thus, the oligonucleotide(s) can be detected and, therefore, the code developed without hybridization or amplification.

Another way of detecting the oligonucleotides of the code without hybridization or amplification and, furthermore, without the oligonucleotides having a different length or hybridization sequence, is to physically or chemically modify one or more of the oligonucleotides. For example, oligonucleotides can be modified to include a molecular beacon. One specific example is the stem-loop beacon where in the absence of hybridization, the oligonucleotide forms a stem-loop structure where the 5′ and 3′ termini comprise the stem, and the beacon (fluorophore, e.g., TMR) located at one termini of the stem is close to the quencher (e.g., DABCYL-CPG) located at the other termini of the stem. In this stem-loop configuration the beacon is quenched and, therefore, there is no emission by the oligonucleotide. When the oligonucleotide hybridizes to a complementary nucleic acid the stem structure is disrupted, the fluorophore is no longer quenched and the oligonucleotide then emits a fluorescent signal (see, e.g., Tan et al., Chem. Eur. J. 6:1107 (2000)). Thus, by including different beacons in oligonucleotides having different emission spectrums, each oligonucleotide containing a unique beacon can be identified by merely detecting the emission spectrum, without amplification or size-fractionation. Another specific example is the scorpion-probe approach, in which the stem-loop structure with the beacon and quencher is incorporated into a primer. When the primer hybridizes to the target oligonucleotide and the target is amplified, the primer is extended unfolding the stem-loop and the loop hybridizes intramolecularly with its target sequence, and the beacon emits a signal (see, e.g., Broude, N. E. Trends Biotechnol. 20:249 (2002)). As the number of beacons expands, the number of unique codes available expands. Thus, beacons in oligonucleotides can be used in combination with other oligonucleotides having a physical or chemical difference of the code, such as a different length.

Additional physical or chemical modifications that facilitate developing the code without amplification or fractionation include radioisotope-labeled nucleotides (e.g., dCTP) and fluorescein-labeled nucleotides (UTP or CTP). Detecting the labels indicates the presence of the oligonucleotide so labeled. The labels may be incorporated by any of a number of means well known to those skilled in the art. For example, the oligonucleotides can be directly labeled without hybridization or amplification or during oligonucleotide amplification, in which case the oligonucleotide(s) primer pairs can be labeled before, during, or following hybridization and subsequent amplification. Typically labeling occurs before hybridization. In a particular example, PCR with labeled primers or labeled nucleotides will produce a labeled amplification product.

“Direct labels” are directly attached to or incorporated into the oligonucleotides prior to hybridization. Alternatively, a label may be attached directly to the primer or to the amplification product after the amplification is completed using methods well known to those of skill in the art including, for example nick translation or end-labeling. Indirect labels are attached to the hybrid duplex after hybridization. For example, an indirect label such as biotin can be attached to the oligonucleotides prior to hybridization. Following hybridization, an avidin-conjugated fluorophore will bind the biotin bearing hybrid duplexes to facilitate detection of the oligonucleotide.

Labels therefore include any composition that can be attached to or incorporated into nucleic acid that is detectable by spectroscopic, photochemical, biochemical, immunochemical, electrical, optical or chemical means such that it provides a means with which to identify the oligonucleotide. Useful labels include biotin for staining with labeled streptavidin conjugate, magnetic beads (e.g, Dynabeads™), fluorescent dyes (e.g., 6-FAM, HEX, TET, TAMRA, ROX, JOE, 5-FAM, R110, fluorescein, texas red, rhodamine, lissamine, phycoerythrin (Perkin Elmer Cetus), Cy2, Cy3, Cy3.5, Cy5, Cy5.5, Cy7, FluorX (Amersham Biosciences; Genisphere, Hatfield, Pa.), radiolabels, enzymes (e.g., horse radish peroxidase, alkaline phosphatase and others used in ELISA), Alexa dyes (Molecular Probes), Q-dots and colorimetric labels, such as colloidal gold or colored glass or plastic beads (e.g., polystyrene, polypropylene, latex, etc.).

When the code is developed in the exemplary illustration (FIGS. 1 and 2), the oligonucleotides are mixed with primer sets. Thus, the invention further provides compositions including a plurality of unique primer pairs (e.g., two or more) and a plurality of oligonucleotides (e.g., two or more) with or without a sample.

The unique primer pairs are within a given primer set. That is, whether or not one or more of the individual oligonucleotides of a code are present, the primer pairs are capable of specifically hybridizing to and amplifying one or more oligonucleotides of the code. If present, oligonucleotides differentiated by size will be amplified and the amplified products will have different lengths. In various embodiments, a composition includes three or more unique primer pairs and two or more oligonucleotides, wherein the unique primer pairs are denoted a first, second, third, fourth, fifth, sixth, etc., primer set, one or more of the unique primer pairs having a different sequence, at least two of the unique primer pairs capable of specifically hybridizing to the two oligonucleotides. The corresponding oligonucleotides to which the primers hybridize are denoted a first, second, third, fourth, fifth, sixth, etc. oligonucleotide set, the oligonucleotides having a length from about 8 nucleotides to 50 Kb, the oligonucleotides in each set having a physical or chemical difference (e.g., a different length) from the other oligonucleotides comprising the same oligonucleotide set. In various aspects, the number of primer pairs in a set is four or more, five or more, six or more unique primer pairs (e.g., seven, eight, nine, ten, 11, 12, 13, 14, 15, 15-20, 20-25, and so on and so forth). In various additional aspects, the number of oligonucleotides is three, four, five, six or more (e.g., seven, eight, nine, ten, 11, 12, 13, 14, 15, 15-20, 20-25, and so on and so forth).

In additional embodiments, compositions include one or more oligonucleotides denoted a second oligonucleotide set, each of the oligonucleotides having a different sequence therein capable of specifically hybridizing to a unique primer pair, the unique primer pair from a second primer set. The second oligonucleotide set includes oligonucleotides incapable of specifically hybridizing to a sample, a length from about 8 nucleotides to 50 Kb, and a physical or chemical difference (e.g., a different length) from the other oligonucleotides within the second oligonucleotide set. In one aspect, one or more oligonucleotides of the second oligonucleotide set have the same length as an oligonucleotide of the first oligonucleotide set. In further embodiments, compositions include one or more oligonucleotides denoted a third oligonucleotide set, each of the oligonucleotides having a different sequence therein capable of specifically hybridizing to a unique primer pair, the unique primer pair from a third primer set. The third oligonucleotide set includes oligonucleotides incapable of specifically hybridizing to a sample, a length from about 8 nucleotides to 50 Kb, and a physical or chemical difference (e.g., a different length) from the oligonucleotides within the third oligonucleotide set. In further aspects, one or more oligonucleotides of the third oligonucleotide set has the same length as an oligonucleotide of the first or second oligonucleotide set.

Invention compositions can include one or more additional oligonucleotide sets (e.g., fourth, fifth, sixth, seventh, eighth, ninth, tenth, etc. sets), the additional oligonucleotide sets each including oligonucleotides within that set having a different sequence therein capable of specifically hybridizing to a unique primer pair from a corresponding primer set (e.g., fourth, fifth, sixth, seventh, eighth, ninth, tenth, etc. sets). Each oligonucleotide within each of the additional oligonucleotide sets is incapable of specifically hybridizing to a sample, has a length from about 8 nucleotides to 50 Kb, and has a physical or chemical difference (e.g., a different length) from the other oligonucleotides within that oligonucleotide set.

As used herein, the term “sample” means any physical entity, which is capable of being coded (bio-tagged) in accordance with the invention. Samples therefore include any material which is capable of having a code associated with the sample. A sample therefore may include non-biological and biological samples as well as samples suitable for introduction into a biological system, e.g., prescription or over-the-counter medicines (e.g., pharmaceuticals), cosmetics, perfume, foods or beverages.

Specific non-limiting examples of non-biological samples include documents, such as letters, commercial paper, bonds, stock certificates, contracts, evidentiary documents, testamentary devices (e.g., wills, codicils, trusts); identification or certification means, such as birth certificates, licensing certificates, signature cards, driver's licenses, identification cards, social security cards, immigration status cards, passports, fingerprints; negotiable instruments, such as currency, credit cards, or debit cards. Additional non-limiting examples of non-biological samples include wearable garments such as clothing and shoes; containers, such as bottles (plastic or glass), boxes, crates, capsules, ampoules; labels, such as authenticity labels or trademarks; artwork such as paintings, sculpture, rugs and tapestries, photographs, books; collectables or historical or cultural artifacts; recording medium such as analog or digital storage medium or devices (e.g., videocassette, CD, DVD, DV, MP3, cell phones); electronic devices such as, instruments; jewelry such as rings, watches, bracelets, earrings and necklaces; precious stones or metals such as diamonds, gold, platinum; and dangerous devices, such as firearms, ammunition, explosives or any composition suitable for preparing explosives or an explosive device.

Specific non-limiting examples of biological samples include foods, such as meat (e.g., beef, pork, lamb, fowl or fish), grains and vegetables; and alcohol or non-alcoholic beverages, such as wine. Non-limiting examples of biological samples also include tissues and whole organs or samples thereof, forensic samples and biological fluids such as blood (blood banks), plasma, serum, sputum, semen, urine, mucus, stool and cerebrospinal fluid. Additional non-limiting examples of biological samples include living and non-living cells, eggs (fertilized or unfertilized) and sperm (e.g., animal husbandry or breeding samples). Further non-limiting examples of biological samples include bacteria, virus, yeast, or mycoplasma, such as a pathogen (e.g., smallpox, anthrax).

Samples that are nucleic acid include mammalian (e.g., human), bacterial, viral, archaea and fungi (e.g., yeast) nucleic acid. As discussed, oligonucleotides used to code such nucleic acid samples do not specifically hybridize to the nucleic acid sample to the extent that the hybridization interferes with developing the code. Thus, for example, where the sample is human nucleic acid, the oligonucleotides typically do not specifically hybridize to the human nucleic acid; where the sample is bacterial nucleic acid, the oligonucleotides typically do not specifically hybridize to the bacterial nucleic acid; where the sample is viral nucleic acid, the oligonucleotides typically do not specifically hybridize to the viral nucleic acid, etc.

The association between the code and the sample is any physical relationship in which the code is able to uniquely identify the sample. The code may therefore be attached to, integrated within, impregnated with, mixed with, or in any other way associated with the sample. The association does not require physical contact between the code and the sample. Rather, the association is such that that the sample is identified by the code, whether the sample and code physically contact each other or not. For example, a code may be attached to a container (e.g., a label on the outside surface of a vial) which contains the sample within. A code can be associated with product packaging within which is the actual sample. A code can be attached to a housing or other structure that contains or otherwise has some association with the sample such that the code is capable of uniquely identifying the sample, without the code actually physically contacting the sample. The code and sample therefore do not need to physically contact each other, but need only have a relationship where the code is capable of identifying the sample.

Oligonucleotides can be added to or mixed with the sample and the mixture can be a solid, semi-solid, liquid, slurry, dried or desiccated, e.g., freeze-dried. Oligonucleotides can be relatively inseparable from the sample. For example, where the oligonucleotides are mixed with a sample that is a biological sample such as nucleic acid, the oligonucleotides are separable from the sample using a molecular biological or, biochemical or biophysical technique, such as size- or affinity based electrophoresis, column chromatography, hybridization, differential elution, etc.

As set forth herein, oligonucleotides can be in a relationship with the sample such that they are easily physically separable from the sample. In the example of a substrate, one or more of the oligonucleotides can be easily physically separable from the sample, under conditions where the sample remains substantially attached to the substrate. For example, when the oligonucleotides are affixed to a dry solid medium (e.g., Guthrie card) and the sample is likewise affixed to the same dry solid medium, the two may be affixed at different positions on the medium. By knowing the position of the oligonucleotides or sample, they can be easily physically separated by removing a section of the substrate to which the oligonucleotides or sample are attached (e.g., a punch). In another example, the oligonucleotides may be dispensed in a well of a multi-well plate (e.g., 96 well plate), with other wells of the plate containing sample(s). The oligonucleotides are physically separated from the sample by retrieving them from the well (e.g., with a pipette) into which they were dispensed.

In either case, whether oligonucleotides of the code physically contact the sample, or the oligonucleotides of the code are associated with but do not physically contact the sample, the oligonucleotides can be identified in order to develop the code. Thus, the invention is not limited with respect to the nature of the association between the oligonucleotides of the code and the sample that is coded.

Substrates to which the oligonucleotides and samples can be synthesized, affixed, attached or stored within or upon include essentially any physical entity or material, such as two dimensional surface, that is permeable, semi-permeable or impermeable, either rigid or pliable and capable of either storing, binding to or having attached thereto or impregnated with oligonucleotides. Substrates that include a sample or oligonucleotide (e.g., code oligonucleotide, identifier oligonucleotide or primer pair) are referred to herein as a “carrier substrate.” Substrates include a plurality of substrates, for example, an archive of two or more substrates.

Substrates include dry solid medium, for example, cellulose, polyester, nylon, glass, plastics (including acrylic, polystyrene, polypropylene, polyethylene, polybutylene, polycarbonate, polyurethanes, etc.), polysaccharides, nitrocellulose, resins, silica or silica-based materials including silicon, polysiloxanes, polyacetates, carbon, metals, inorganic glasses and mixtures thereof etc. Typically, the substrate is flat (planar), although other configurations of substrates may be employed, for example, three dimensional materials such as beads and microspheres. Substrates can be of any size or dimension. A typical planar substrate has a surface area of less than about 4 square centimeters.

Specific commercially available dry solid medium includes, for example, Guthrie cards, IsoCode (Schleicher and Schuell), and FTA (Whatman). A medium having a mixture of cellulose and polyester is useful in that low molecular weight nucleic acid (e.g., the oligonucleotides comprising the code) preferentially binds to the cellulose component and high molecular weight nucleic acid (e.g., genomic DNA) preferentially binds to the polyester component. A specific example of a cellulose/polyester blend is LyPore SC (Lydall), which contains about 10% cellulose fiber and 90% polyester. Washing the dry solid medium with an appropriate liquid or removing a section (e.g., a punch) retrieves the oligonucleotides or sample from the medium, which can subsequently be analyzed to develop the code or to analyze the sample.

Substrates include foam, such as an absorbent foam. In the particular example of a sponge-like absorbent foam having oligonucleotides or sample, the foam can be wet or wetted with an appropriate liquid, and squeezed or centrifuged to release liquid containing the oligonucleotides or sample. Substrates include structures having sections, compartments, wells, containers, vessels or tubes, separated from each other to prevent mixing of samples with each other or with the oligonucleotides. Multi-well plates, which typically contain 6 to 1000 wells, are one particular non-limiting example of such a structure.

Substrates also include two- or three-dimensional arrays that include biological molecules or materials, which are referred to herein as “target molecules,” “target sequences,” or “target materials.” Such substrates are useful for sample screening, sequencing, mapping, fingerprinting and genotyping. The particular identity of biological molecules included may be known or unknown. For example, a known nucleic acid sequence will specifically hybridize to a complementary sequence and, therefore, such a sequence has a defined recognition specificity.

Biological molecules may be naturally-occurring or man-made. Biological molecules typically include functional groups that participate in interaction with proteins, particularly hydrogen bonding, and typically include at least an amine, carbonyl, hydroxyl or carboxyl group. Cyclical carbon or heterocyclic structures or aromatic or polyaromatic structures substituted with one or more of the above functional groups may also be included. Thus, a particular example of a biological molecule is a small organic compound having a molecular weight of less than about 2,500 daltons, for example, a drug. Additional particular examples of biological molecules include nucleic acids, proteins (antibodies, receptors, ligands), saccharides, carbohydrates, lectins, fatty acids, lipids, steroids, purines, pyrimidines, derivatives, structural analogs and combinations thereof.

A “probe” is a molecule that potentially interacts with a target molecule, sequence or material, e.g., a query such as a nucleic acid or protein sample. Thus, target molecules, sequences and materials can be referred to as “anti-probes.” As with a target molecule, a probe is essentially any biological molecule or a plurality of such molecules.

Substrates can include any number of biological molecules. For example, arrays with nucleic acid or protein sequences greater than about 25, 50, 100, 1000, 10,000, 100,000, 1,000,000, 10,000,000, 100,000,000, 1,000,000,000, or more are known in the art. Such substrates, also referred to as “gene chips” or “arrays,” can have any nucleic acid or protein density; the greater the density the greater the number of sequences that can be screened on a given chip. Thus, very low density, low density, moderate density, high density, or very high density arrays can be made. Very low density arrays are less than 1,000. Low density arrays are generally less than 10,000, with from about 1,000 to about 5,000 being preferred. Moderate density arrays range from about 10,000 to about 100,000. High density arrays range about 100,000 to about 10,000,000. A typical array density is at least 25 molecules per square centimeter. In some arrays, multiple substrates may be used, either of different or identical biological molecules. Thus, for example, large arrays may comprise a plurality of smaller arrays or substrates.

Arrays typically have a surface with a plurality of biological molecules located at pre-determined or positionally distinguishable (addressable) locations so that any interaction (e.g., hybridization) between a target molecule and a probe can be detected. The biological molecules may be in a pattern, i.e., a regular or ordered organization or configuration, or randomly distributed. An example of a regular pattern are sites located in an X-Y, or “row” x “column” coordinate plane (i.e., a grid pattern). A “pattern” refers to a uniform or organized treatment of substrate, as described above, or a uniform or organized spatial relationship among the target molecules attached to the substrate, resulting in discrete sites.

Appropriate methods to detect interactions depend on the nature of the target and probe. Exemplary methods are known in the art and include, for example, radionuclides, enzymes, substrates, cofactors, inhibitors, magnetic particles, heavy metal and spectroscopic labels. High resolution and high sensitivity detection and quantitation can be achieved with fluorophores and luminescent agents, as set forth herein and known in the art. Hybridization signal detection methods, and methods and apparatus for signal detection and processing of signal intensity data are described, for example, in WO 99/47964 and U.S. Pat. Nos. 5,143,854, 5,547,839, 5,578,832; 5,631,734; 5,800,992, 5,834,758; 5,856,092, 5,902,723, 5,936,324; 5,981,956; 6,025,601; 6,090,555, 6,141,096; 6,185,030; 6,201,639; 6,218,803 and 6,225,625; and U.S. Patent Publication Nos. 20030215841 and 20030073125.

Biological molecules such as nucleic acid or protein (e.g., one or more sample(s)) are typically synthesized on the substrate or are attached to the surface of the substrate (e.g., via a covalent or non-covalent bond or chemical linkage, directly or via an attachment moiety or absorption, or photo-crosslinking) at defined locations (addresses) that are optionally pre-determined. The location of each molecule is typically positionally defined and located at physically discrete individual sites.

The surface of a substrate may be modified such that discrete sites are formed that only have a single type of biological molecule, e.g., a nucleic acid or polypeptide with a particular sequence. For example, the substrate can have a physical configuration such as a wells or small depressions that retain the biological molecule. Wells or small depressions in the substrate surface can be produced using a variety of techniques known in the art, including, for example, photolithography, stamping, molding and microetching techniques.

The substrate may be chemically altered to attach, either covalently or non-covalently, the biological molecules. Exemplary modifications include chemical, electrostatic, hydrophobic and hydrophilic functionalized sites, and adhesives. Chemical modifications include, for example, addition of chemical groups such as amino, carboxy, oxo and thiol groups that can be used to covalently attach biological molecules; addition of adhesive for binding biological molecules; addition of a charged group for the electrostatic attachment of biological molecules; addition of chemical functional groups that renders the sites differentially hydrophobic or hydrophilic so that the substrate associates with the biological molecules on the basis of hydroaffinity.

Array synthesis methods are described, for example, in WO 00/58516, WO 99/36760, and U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752; and U.S. Patent Publication Nos. 20040023367, 20030157700 and 20030119011. Nucleic acid arrays useful in the invention are commercially available from Illumina (San Diego, Calif.) and Affymetrix (Santa Clara, Calif.).

Substrates that include a two- or three-dimensional array of biological molecules, such as nucleic acid or protein sequences, and individual nucleic acid or protein sequences therein, may be coded in accordance with the invention. Thus, for example, the substrate itself can be the sample, in which case a substrate containing a plurality of nucleic acid or protein sequences will have a unique code. Alternatively, one or more of each individual nucleic acid or protein sequence on the substrate can have an individual code. For example, a unique oligonucleotide code can be added to one or more samples on the substrate in order to uniquely identify the coded samples.

In another alternative, a substrate can include oligonucleotides, referred to as identifier oligonucleotides, that identify the code in the sample. For example, in micro-array technology, typically a biological sample is contacted with an array that contains target molecules that potentially interact with probe molecules (e.g., protein or nucleic acid) within that sample. A profile of the sample is generated, for example, a gene expression profile, based upon the particular targets that interact with the probes in the sample. Arrays that include “identifier oligonucleotides,” which are oligonucleotides capable of specifically hybridizing to oligonucleotides of the code, can determine the code in the sample analyzed with the array. The identifier oligonucleotides are of sufficient number that collectively they are capable of specifically hybridizing to every possible code oligonucleotide that may be present in the sample. Specific hybridization between an identifier oligonucleotide and a code oligonucleotide identifies the oligonucleotides that are present in the code, by producing a signal (e.g., fluorescence, chemiluminesence) that indicates such hybridization. In contrast, identifier oligonucleotides that do not specifically hybridize to any code oligonucleotides do not produce a signal indicative of hybridization, indicating that the corresponding complementary code oligonucleotides are absent from the sample.

Each identifier oligonucleotide is immobilized at a pre-determined location or position on a substrate (e.g., an array). For example, identifier oligonucleotides can be positioned at specified addresses on an array in a pattern or other configuration such as a row or a column, or a section of rows and columns of an array, such as in a “row×column” pattern of 2×2 (4 identifier oligonucleotides), 2×3 or 3×2 (6 identifier oligonucleotides), 3×3 (9 identifier oligonucleotides), 3×4 or 4×3 (12 identifier oligonucleotides), 4×4 (16 identifier oligonucleotides), 4×5 or 5×4 (20 identifier oligonucleotides), 5×5 (25 identifier oligonucleotides), etc. As with the oligonucleotides of the code, the identifier oligonucleotides also do not specifically hybridize to nucleic acids of the sample to the extent that such hybridization interferes with developing the code.

Samples coded with a unique combination of oligonucleotides in accordance with the invention can contact a substrate (e.g., an array) that includes such identifier oligonucleotides. Following contacting with the coded sample, identifier oligonucleotides that specifically hybridize to their complementary code oligonucleotides present in the sample are detected. As before, the code is identified or “decoded” based upon which oligonucleotides are present in the code (positive) and which oligonucleotides are absent (negative). As before, the presence and absence of a given oligonucleotide of the code can optionally be represented for each position as in a bar-code, for example, “1” to indicate hybridization to the particular identifier oligonucleotide, and “0” to indicate the absence of hybridization to the particular identifier oligonucleotide.

Using substrates including such identifier oligonucleotides allows the sample profile to be developed with the sample code, which provides an internal check of sample identity. In other words, the sample code and, therefore, the identity of the sample is permanently linked to and associated with the profile for that sample.

The invention therefore further provides compositions including a substrate, and a plurality of polynucleotide or polypeptide sequences each immobilized at pre-determined positions on the substrate. In one embodiment, at least two of the polypeptide or polynucleotide sequences are designated as target sequences and are distinct from each other, and at least one polynucleotide sequence is designated as an identifier oligonucleotide that does not specifically hybridize to a nucleic acid that is capable of specifically hybridizing to the target sequences. In another embodiment, at least two polynucleotide sequences, designated as target sequences are distinct from each other, and at least a third polynucleotide sequence designated as an identifier oligonucleotide does not specifically hybridize to a nucleic acid that is capable of specifically hybridizing to the target sequences. In various aspects, the target sequences comprises a library (e.g., a nucleic acid, such as a genomic, cDNA or EST; or a polypeptide library, such as a binding molecule, for example, an antibody, receptor, receptor binding ligand or a lectin, or an enzyme library), for example, a mammalian library having at least 10 to 100, 100 to 1000, 1000 to 10,000, 10,000, to 100,000, or more target sequences.

The number of identifier oligonucleotides can vary and need only be sufficient to identify every oligonucleotide potentially present in a code or bio-tag. Thus, there can be between 2 and 5 identifier oligonucleotides, or more, as appropriate for specific hybridization to the code oligonucleotides, for example, between 5 and 10, 10 and 15, 15 and 20, 20 and 25, 25 and 30, 30 and 50, or more identifier oligonucleotides. When present on a substrate or array, the identifier oligonucleotides typically are patterned, for example, in a column or a row, to permit ease of identification.

As with oligonucleotides of a code or bio-tag, when the sample includes nucleic acid the identifier oligonucleotides are not capable of specific hybridization to the nucleic acid, to the extent that such hybridization prevents the code form being developed. As with code oligonucleotides, such hybridization can be minimized using code and corresponding identifier oligonucleotides that are not the same species as the sample target sequences. For example, where the sample target sequences are human, code oligonucleotides and, therefore, identifier oligonucleotides are not fully human; where the sample target sequences are plant, code oligonucleotides and, therefore, identifier oligonucleotides are not fully plant; where the sample target sequences are bacterial, code oligonucleotides and, therefore, identifier oligonucleotides are not fully bacterial; where the sample target sequences are viral, code oligonucleotides and, therefore, identifier oligonucleotides are not fully viral; etc.

Samples containing code oligonucleotides can be contacted directly to such substrates or can be processed prior to contacting the substrate. For example, if it is desired to increase the amount of sample or code prior to contact with the substrate, the code or sample can be amplified. Thus, for a nucleic acid sample, if desired, amounts of both the nucleic acid and the code can be increased to increase hybridization sensitivity or hybridization detection and, therefore, detection of low copy number nucleic acid sequences or code oligonucleotides with the substrate.

As described herein, code oligonucleotides can be designed that have a common primer set but differ in the internal sequence between the primer binding sites or the sequence(s) that flank the primer binding sites. In this way, all code oligonucleotides in a sample can be amplified with a single primer set. Since the code oligonucleotide includes a unique sequence, a specifically hybridizing identifier oligonucleotide can be designed which has a sequence that is complementary to the unique sequence of the code oligonucleotide. For example, differing intervening sequences between the primer-binding site of two code oligonucleotides allow them to be distinguished from each other, even though both code oligonucleotide have the same sequences for primer binding. This design can increase the number of codes that can be produced for a given set of primers.

An additional feature of this aspect of the invention is that a code oligonucleotide can be used to provide highly specific information. For example, a code oligonucleotide could be assigned to a particular hospital, clinic, research institution, or any other source from which a sample was obtained. The assigned code would be unique to the source of the sample such that the code positively identifies the sample source (e.g., the particular hospital, clinic, etc., to which the code is assigned). Such a code oligonucleotide would provide a link between the sample and the source thereby providing a means to trace the sample to its source and minimizing sample misidentification. A code oligonucleotide could be used to identify a particular substrate, array or study type. The information that the code provides is therefore not limited to binary information. In addition, the position of an oligonucleotide on a substrate or array could also be used to provide information.

Sample identification afforded by including a unique bio-tag as set forth herein, and optionally including identifier oligonucleotides on an array or substrate that may be used for sample analysis, allows tracking of the sample at any time. The ability to positively identify a sample based upon its unique code prevents errors due to sample mishandling, mislabeling or misidentification that can occur during procedures employing the sample. Positive sample identification is particularly valuable where large numbers of samples are processed, where sample misidentification can lead to erroneous data, and where samples are subject to multiple studies or procedures. For example, genotyping studies typically require analysis of large numbers of samples in order to detect associations between a disease and a gene loci. Positive sample identification is crucial since even low error rates (from 1-2%) can have a significant impact, increasing both Type I (false positives) and Type II (loss of power) errors. Sample swap, in which one sample is mislabeled, misidentified, or mishandled as another sample, is a well-known source of error in genotyping studies. The invention, which, inter alia, provides compositions and methods for producing uniquely identified samples as well as compositions and methods for identifying such samples, can be employed to reduce and eliminate such errors.

The invention provides kits including compositions as set forth herein. In one embodiment, a kit includes two or more oligonucleotides in one or more oligonucleotide sets, packaged into suitable packaging material. Kits can contain oligonucleotide(s) of one or more sets, primer pair(s) of one or more sets, optionally alone or in combination with each other. A kit typically includes a label or packaging insert including a description of the components or instructions for use (e.g., coding a sample). A kit can contain additional components, for example, primer pairs that specifically hybridize to the oligonucleotides.

The term “packaging material” refers to a physical structure housing the components of the kit. The packaging material can maintain the components sterilely, and can be made of material commonly used for such purposes (e.g., paper, corrugated fiber, glass, plastic, foil, ampoules, etc.). The label or packaging insert can include appropriate written instructions, for example, practicing a method of the invention. Kits of the invention therefore can additionally include labels or instructions for using the kit components in a method of the invention. Instructions can include instructions for practicing any of the methods of the invention described herein. The instructions may be on “printed matter,” e.g., on paper or cardboard within the kit, or on a label affixed to the kit or packaging material, or attached to a vial or tube containing a component of the kit. Instructions may additionally be included on a computer readable medium, such as a disk (floppy diskette or hard disk), optical CD such as CD- or DVD-ROM/RAM, DV, MP3, magnetic tape, electrical storage media such as RAM and ROM and hybrids of these such as magnetic/optical storage media.

Invention kits can include each component (e.g., the oligonucleotides) of the kit enclosed within an individual container and all of the various containers can be within a single package. Invention kits can be designed for long-term, e.g., cold storage.

The invention provides methods of producing samples that are coded (i.e., “bio-tagged”) in order to identify the sample. In one embodiment, a method includes: selecting a combination of two or more oligonucleotides to add to the sample which are incapable of specifically hybridizing to the sample, each having a length from about 8 to 50 Kb nucleotides and a physical or chemical difference (e.g., a different length), and one or more having a different sequence therein capable of specifically hybridizing to a unique primer pair; and adding the combination of two or more oligonucleotides to the sample. The combination of oligonucleotides identifies the sample and, therefore, the method produces a bio-tagged sample. In additional embodiments, a method of the invention employs one or more oligonucleotides from multiple (e.g., two, three, four, five, six, seven, eight, nine, ten, etc., or more) oligonucleotide sets in which one or more oligonucleotides from the additional oligonucleotide sets is added to the sample. In one particular embodiment, one or more oligonucleotides from a second set is added, one or more of the oligonucleotide(s) of the second set having a different sequence therein capable of specifically hybridizing to a unique primer pair of a second primer set, incapable of specifically hybridizing to the sample, a physical or chemical difference (e.g., a different length) from the other oligonucleotides of the second set, and a length from about 8 to 50 Kb nucleotides. In another particular embodiment, one or more oligonucleotides from a third oligonucleotide set is added, one or more of the oligonucleotide(s) of the third set having a different sequence therein capable of specifically hybridizing to a unique primer pair of a third primer set, incapable of specifically hybridizing to the sample, a physical or chemical difference (e.g., a different length) from the other oligonucleotides of the third set and a length from about 8 to 50 Kb nucleotides. In one aspect of the methods of producing a coded sample, one or more of the oligonucleotides of the code is physically separated or separable from the sample.

The invention also provides methods of identifying a coded (i.e., “bio-tagged”) sample. In one embodiment, a method includes: detecting in a sample the presence or absence of two or more oligonucleotides, wherein the oligonucleotides are identified based upon a physical or chemical difference (e.g., length), thereby identifying a combination of oligonucleotides in the sample; comparing the combination of oligonucleotides to a database of particular oligonucleotide combinations known to identify particular samples; and identifying the sample based upon which of the particular oligonucleotide combinations in the database is identical to the combination of oligonucleotides in the sample. The oligonucleotide combination can be identified based upon a primer or primer pair(s) that specifically hybridizes to the oligonucleotides, e.g., differential primer hybridization with or without subsequent amplification. Thus, in another embodiment, a method further includes specifically hybridizing one or more unique primer pairs of one or more primer sets to the oligonucleotides that may be present thereby identifying oligonucleotide(s) present. Oligonucleotides are identified based upon primer pair(s) hybridization to the oligonucleotides that are present; the combination of particular oligonucleotides present in the sample is the code of the sample. Methods for identifying/detecting the oligonucleotides include hybridization to two or more unique primer pairs having a different sequence; and hybridization to two or more unique primer pairs having a different sequence and subsequent amplification (e.g., PCR). In further aspects, oligonucleotides that are likely to be present in the sample are selected from two or more oligonucleotide sets (e.g., two, three, four, five, six, seven, eight, nine, etc. sets) and, as such, a method of the invention can additionally include specifically hybridizing one or more unique primer pairs of two or more primer sets to the oligonucleotides that may be present with or without subsequent amplification in order to identify which of the oligonucleotides from the different oligonucleotide sets are present.

The invention further provides archives of coded (i.e., bio-tagged) sample(s). In one embodiment, an archive of bio-tagged samples includes: one or more samples; two or more oligonucleotides incapable of specifically hybridizing to one or more of the samples, the oligonucleotides each having a physical or chemical difference (e.g., a different length), and a length from about 8 to 50 Kb nucleotides, one or more of the oligonucleotides having a different sequence therein capable of specifically hybridizing to a unique primer pair, in a unique combination that identifies the one or more samples; and a storage medium for storing the sample(s). In various aspects, an archive includes 1 to 10, 10 to 50, 50 to 100, 100 to 500, 500 to 1000, 1000 to 5000, 5000 to 10,000, 10,000 to 100,000, or more samples, one or more of which is coded.

The invention further provides methods of producing archives of coded (i.e., bio-tagged) samples. In one embodiment, a method includes: selecting a combination of two or more oligonucleotides that are incapable of specifically hybridizing to the sample, each having a chemical or physical difference (e.g., a different length), and a length from about 8 to 50 Kb nucleotides, and one or more of the oligonucleotides having a different sequence therein capable of specifically hybridizing to a unique primer pair; and adding the combination of two or more oligonucleotides to a sample. The bio-tagged sample produced is then placed in a storage medium.

Two or more samples placed in a storage medium comprise an archive. Substrates can also be included in an archive, which includes a storage medium for the substrate. Such substrates can contain a sample, a code or bio-tag, one or more identifier oligonucleotides, etc., as described herein.

The invention additionally provides methods of identifying a sample code using an array or substrate that includes one or more identifier oligonucleotides. In one embodiment, a method includes providing a substrate including two or more identifier oligonucleotides, wherein the number of identifier oligonucleotides are sufficient to specifically hybridize to all oligonucleotides potentially present in a coded sample; contacting the substrate with a coded sample; and detecting specific hybridization between the identifier oligonucleotides and code oligonucleotides that are present in the sample, thereby identifying the code oligonucleotides present in the sample. Comparing the combination of code oligonucleotides with a database including particular oligonucleotide combinations known to identify particular samples identifies the sample based upon the particular oligonucleotide combination in the database that is identical to the combination of oligonucleotides in the sample. In one aspect, the oligonucleotides of the code are amplified prior to contacting the coded sample with the substrate or array.

The invention moreover provides methods of producing substrates and arrays capable of identifying a sample code. In one embodiment, a method includes selecting a combination of two or more identifier oligonucleotides to add to substrate, the identifier oligonucleotides each capable of specifically hybridizing to a corresponding code oligonucleotide; and adding the combination of two or more identifier oligonucleotides to the substrate, wherein the number of identifier oligonucleotides are sufficient to specifically hybridize to all oligonucleotides potentially present in a coded sample. Typically, the identifier oligonucleotides are selected on the basis of the code oligonucleotide sequences in order to ensure specific hybridization and, therefore, code identification.

In various aspects, between 2 and 5, 5 and 10, 10 and 15, 15 and 20, 20 and 25, 25 and 30, 30 and 50, or more identifier oligonucleotides are present on the substrate or array. In additional aspects, the substrate or array includes a check code or another oligonucleotide that provides other information (e.g., the source of the sample, such as the hospital or clinic from which it originated). In yet additional aspects, the identifier oligonucleotides are located in pre-determined positions (addresses) on the array or substrate, for example, in an ordered pattern such as a column or a row.

Methods of producing archives of substrates and arrays capable of identifying a sample code are also provided. In one embodiment, a method includes selecting a combination of two or more identifier oligonucleotides to add to a substrate, the identifier oligonucleotides each capable of specifically hybridizing to a corresponding code oligonucleotide; adding the combination of two or more identifier oligonucleotides to the substrate, wherein the number of identifier oligonucleotides are sufficient to specifically hybridize to all oligonucleotides potentially present in a coded sample; and placing the substrate or array in a storage medium.

It will be appreciated that some or all of the foregoing functional aspects related to creating bio-tagged samples and to “reading” or otherwise interpreting bio-tags to identify specific samples with particularity may be facilitated by one or more automated systems operative under computer or microprocessor control. In that regard, a computer executed method of producing a bio-tag for a sample, as well as a computer executed method of applying a bio-tag to a sample carrier, may generally utilize a processing component having sufficient capabilities and processing bandwidth to enable the functionality set forth below with specific reference to FIGS. 2-5. Such a processing component may be embodied in or comprise a computer, a microcomputer or microcontroller, a programmable logic controller, one or more field programmable gate arrays, or any other individual hardware element or combination of elements having utility in data storage and processing operations as generally known in the art or developed and operative in accordance with known principles.

Specifically, the term “processing component” in this context generally refers to hardware, firmware, software, or more specifically, to some combination thereof, appropriately configured, suitably programmed, and generally operative to execute computer readable instructions encoded on a recording medium and causing an apparatus executing the instructions to create, read, or otherwise to utilize bio-tag codes as set forth with particularity herein. In that regard, a processing component may additionally provide partial or complete instruction sets to various types of automated apparatus, robotic systems, and other computer controllable devices, and may be operative to communicate with, receive feedback from, and dynamically influence operation of independent processing components or electronic elements associated or integrated with such apparatus.

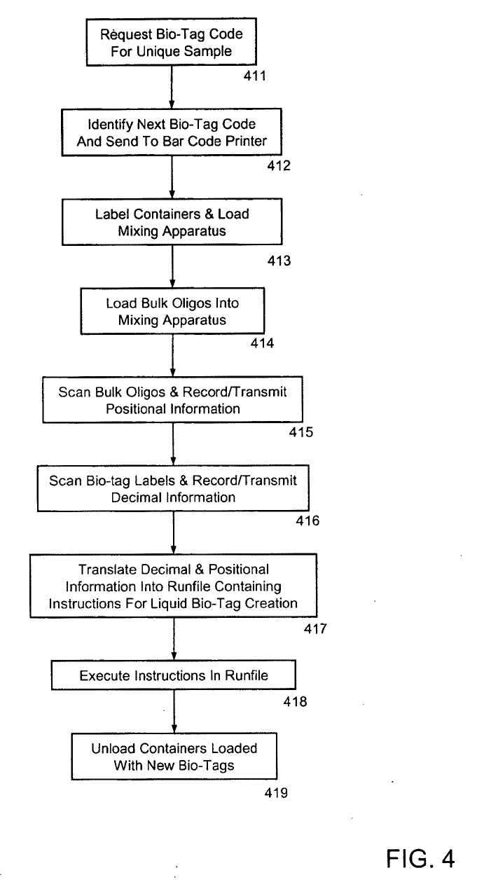

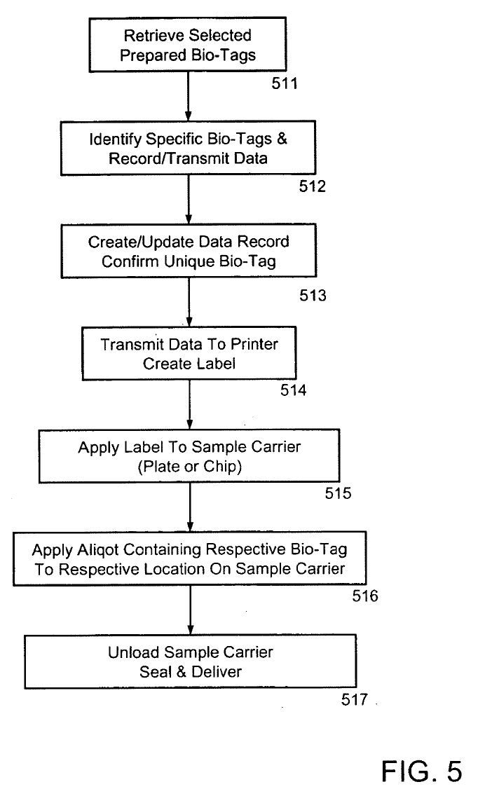

In that regard, it will be appreciated that a computer readable medium encoded with data and instructions for producing a bio-tagged sample may readily cause an apparatus executing the instructions to select a unique combination of oligonucleotides to add to the sample as described in detail below; data records regarding unique combinations of oligonucleotides may be maintained in a database or other data structure accessible by a computer or processing component and may enable the functionality set forth below with specific reference to FIGS. 4 and 5. As described in detail above with specific reference to FIGS. 1A and 1B, the oligonucleotides may be selected such that each is incapable of specifically hybridizing to the sample. Additionally, the oligonucleotides may be selected such that each may have a length from about 8 to about 5000 nucleotides, and each may have certain selected physical or chemical properties; in particular, one or more of the oligonucleotides each have a different sequence therein capable of specifically hybridizing to a unique primer pair or to an identifier oligonucleotide as described above. As set forth in more detail below, computer executable instruction sets may cause automated apparatus or robotic devices to contact a unique combination of oligonucleotides with a sample, or with a specified or predetermined well in, or a specified or predetermined location on, a sample carrier. A specified unique combination of oligonucleotides selected by a processing component may be associated with and identify a specified location on the sample carrier, thereby producing a bio-tagged sample or a bio-tagged location on the sample carrier. Data records associating each unique combination of oligonucleotides with each unique bio-tagged sample or location on the sample carrier may be maintained, for example, in the database or other suitable data structure mentioned above.

Further, a computer readable medium encoded with data and instructions for identifying a bio-tagged sample may enable an apparatus executing the instructions to detect in a sample the presence or absence of two or more oligonucleotides; as contemplated herein, the oligonucleotides may generally be identified based upon a physical or chemical difference. Accordingly, automated apparatus may identify a specific unique combination of oligonucleotides in the sample; this functionality may be embodied in or incorporate various automated detection technologies generally known in the art of sample analysis. The computer readable medium may cause an apparatus to compare the unique combination of oligonucleotides with a database comprising data records of particular oligonucleotide combinations known to identify respective particular samples, and to identify an otherwise unknown sample based upon a comparison of the data records and the unique combination of oligonucleotides in the unknown sample.

In accordance with the detailed description provided above, it will be appreciated that a computer readable medium encoded with data and instructions for producing an archive of bio-tagged samples may cause or enable an apparatus executing the instructions to select a unique combination of oligonucleotides to associate with a sample; the oligonucleotides may be selected automatically by an appropriately programmed processing component, and may be selected in accordance with the structural and chemical considerations set forth above with reference to FIGS. IA and 1B. Automated devices operating under control of a processing component may contact the unique combination of oligonucleotides with the sample such that the unique combination of oligonucleotides identifies the sample, thereby producing a bio-tagged sample; similarly, automated or semi-automated devices operating under control of the processing component may place the bio-tagged sample in a storage medium archive facility for storing the bio-tagged sample, and may additionally create a data record associating the storage medium and the storage location with the bio-tagged sample.

FIG. 2A is a simplified diagram illustrating a code generated following size-based fractionation via gel electrophoresis and indicating an alternative convention for reading the code. FIG. 2B is a simplified diagram illustrating the binary code read in accordance with the convention indicated in FIG. 2B. Specifically, each lane of the gel represented in FIG. 2A may be read in sequence (i.e., lane 1, followed by lane 2, followed by lane 3, and so forth) and from bottom to top (i.e., in the direction of increasing base-pair size in FIG. 2A). The binary code in FIG. 2B represents the encoded information extracted when the gel is read in the foregoing manner. Various apparatus and methodologies may be employed for reading results of an electrophoresis gel; the present disclosure is not intended to be limited to any particular technology employed to acquire data from such an electrophoresis operation. Similarly, the conventions employed for encoding data in the gel and for reading or otherwise interpreting same are susceptible of numerous modifications, none of which affect the scope and contemplation of the present disclosure.

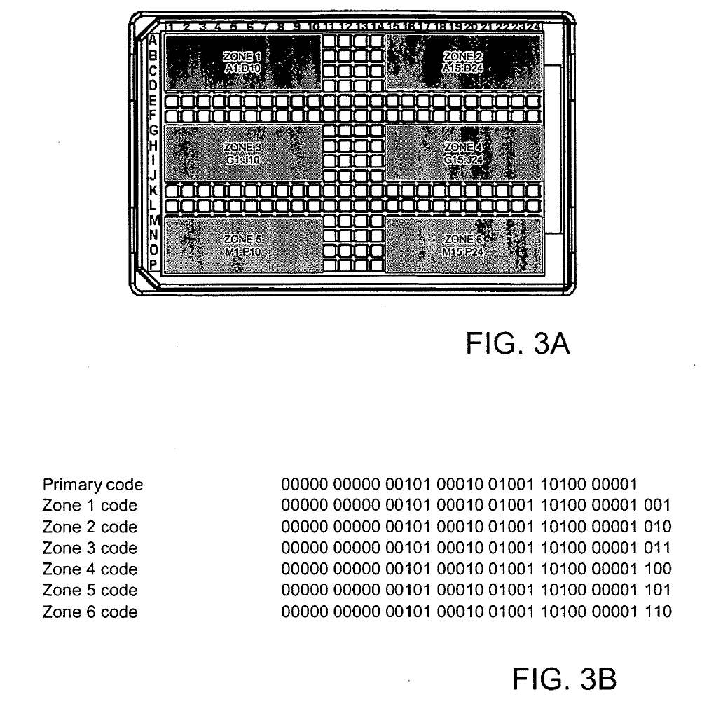

As described herein, various systems and methods of spotting, loading, bio-tagging, or otherwise manipulating samples and sample carriers are described. In that regard, FIG. 3A is a simplified diagram illustrating one embodiment of a sample carrier, and FIG. 3B is a simplified diagram illustrating an exemplary code associated with one bio-tag maintained at different locations on the sample carrier of FIG. 3A.

In some embodiments, a sample carrier may generally be embodied in or comprise a multi-well plate. The plate may employ 384 discrete wells, for example, as illustrated in the FIG. 3A implementation; other plate formats, including 96 wells, for example, are also commonly used. In alternative embodiments, a sample carrier may be embodied in or comprise a bio chip, array, or other substrate, for example, and may generally include a grid or similar coordinate system. Whether such a coordinate system comprises, for example, numbered columns and lettered rows of wells as in the FIG. 3A embodiment, or some other coordinate convention used in conjunction with a multi-well plate or with respect to an array, the coordinate system may facilitate organization of a sample carrier and identification of samples by specifying or uniquely designating a plurality of addressable locations, each of which may contain or support a discrete sample.

The sample carrier of FIG. 3A is further organized or sub-divided into six distinct zones: zone 1 comprises wells at grid locations A 1 through D10; zone 2 comprises wells at grid locations A15 through D24; and so forth. The represented organization is arbitrary and may be selectively altered to accommodate more or fewer zones as desired, i.e., any number or arrangement of different zones or distinct areas on the sample carrier may be established at any convenient location. Similarly, an array, or even a rack of test tubes, may be selectively sub-divided or otherwise organized into zones as desired or required. As indicated in FIG. 3B, a single bio-tag code (such as that representing the bio-tag considered in FIGS. 2A and 2B, in this example) may be used multiple times and still enable unique identification of a discrete sample where a zone designator code or other indicia is appended to the code. For example, a binary suffix “011” appended to the code may be interpreted as an indication that the bio-tag is associated with or located in zone 3 of the sample carrier, whereas the code for the same bio-tag maintained at or located in zone 4 may include a binary suffix “100.” In the foregoing manner, it is possible to employ a single bio-tag up to six different times in conjunction with the exemplary sample carrier of FIG. 3A while allowing or enabling six distinct codes therefor.

FIG. 4 is a simplified flow diagram illustrating the general operation of one embodiment of a method of producing a bio-tag for use in identifying a sample. In accordance with the exemplary FIG. 4 embodiment, a method of producing a bio-tag for a sample may generally begin with a request that a bio-tag be created for a unique sample as indicated at block 411. As contemplated at block 411, an operator or user may login to a software application (such as a Java script, for example, or such as may be embodied in a commercial or proprietary software program) enabled by or running on a processing component asset forth above. Upon login and appropriate operator authentication procedures (such as are generally known in the art), an operator may request a specific number of bio-tags, each of which may be employed to identify a unique sample.

As indicated at block 412, the next available bio-tag code (such as in a predetermined or prerecorded sequence, for example) may be identified and sent to a barcode label printer; in some implementations using decimal format, code 128 barcodes may be employed. In some embodiments, the operation depicted at block 412 may be executed automatically under control of a processing component as set forth above; in such automated implementations, the foregoing software application may query a database or other data structure (such as an ORACLE™ database or other proprietary data archival mechanism) to retrieve a next unique bio-tag available in a particular reference system or bio-tag code universe. In that regard, it will be appreciated that different entities or different archive systems may have one or more bio-tags in common; in this context, however, such common codes may nevertheless be unique in each individual system. Alternatively, an archive or entity identifier segment or sequence may be appended to each bio-tag created, making even repeated sequences or combinations of bio-tag oligonucleotides distinct between entities or archival systems.

The newly-ascertained unique bio-tag code may be transmitted or otherwise communicated to a conventional barcode printer responsive to appropriate command or control signals issued by the processing component. Alternatively, an operator may consult one or more look-up or reference tables, spreadsheet cells, or other archival records to ascertain which of a plurality of bio-tag codes in a particular reference system have not been used, and may send same to a barcode printer manually, or at least partially in accordance with operator intervention. Specifically, it will be appreciated that the operations at blocks 411 and 412 may be at least partially conducted manually or otherwise in conjunction with operator input. In a fully automated embodiment, the processing component may control all operations; additionally or alternatively, the processing component may work in conjunction with independent processing components or programming instruction sets resident in or associated with, for example, the barcode printing apparatus or other automated devices.

As indicated at block 413, barcode labels may be applied to one or more containers, which may then be loaded into a mixing apparatus. It will be appreciated that the identification functionality contemplated at blocks 412 and 413, while described with reference to barcode labels, may alternatively be implemented in accordance with any of various types of identification methodologies. One- and two-dimensional barcodes may have particular utility in that regard, especially when employed in conjunction with automated optical systems or machine reading apparatus. In accordance with some exemplary embodiments, any type of identifying indicia, including alpha-numeric and other coding schemes, may be employed in addition, or as an alternative, to barcode indicia.

As with the operations at blocks 411 and 412, the functionality illustrated at block 413 may be performed automatically through appropriately manipulated automated or robotic apparatus, for example, under control of a processing component; alternatively, the foregoing functions may be executed partially or entirely manually by an operator. In particular, an operator may apply the barcode labels to empty containers and load labeled containers into a mixing apparatus or other device for receiving bio-tag materials or solutions. With respect to the operation depicted at block 413, “containers” may be embodied in, but are not limited to, for example, test tubes, multi-well plates (such as those containing 96, 384, or any other number of discrete wells), or arrays or other suitable substrates, such as generally known and employed in the art of biological and non-biological sample analysis technologies. In some embodiments, an automated liquid handling device for loading bio-tag materials or solutions into containers or onto container media under control of a processing component may be embodied in or comprise a Microlab Star liquid handler apparatus currently available from Hamilton Company, though other single and multiple arm liquid handling systems are generally known in the art and may be suitably configured and programmed to provide the functionality set forth herein.

As indicated at block 414, bulk oligonucleotides may be loaded into the mixing apparatus. Again, this operation may be executed either by an operator, for instance, or entirely or partially under control of a suitably programmed processing component operative to manipulate automated or robotic handling mechanisms. In that regard, and in accordance with some automated or semi-automated embodiments, each particular bulk oligonucleotide may be uniquely identified by a fixed barcode or other indicia on its container, allowing or enabling precise identification of same by various types of mechanical, optical, or electromechanical devices.

As indicated at block 415, the mixing apparatus may scan each bulk oligonucleotide container and send positional information (for each bulk oligonucleotide) to mixer controlling software. The foregoing scanning operation may be conducted independently by the mixing apparatus; additionally or alternatively, some instructions or a complete instruction set regarding desired scanning procedures or parameters may be transmitted by an independent processing component such as set forth above. Similarly, the aforementioned mixing control software may be resident at the mixing apparatus, for example, or may be dynamically or selectively controlled or otherwise influenced by control signals or command instructions transmitted or otherwise communicated from such an external or independent processing component. As indicated at block 416, the mixing apparatus may additionally scan the bio-tag label or labels, and send decimal information to the mixer controlling software; in this context, the decimal information may generally be related to, or indicative of, the specific container (such as a particular well of a multi-well plate) or medium coordinate location to which each bulk oligonucleotide is intended to be supplied.

As indicated at block 417, the control software, independently or in conjunction with data and instructions received from a processing component, may then translate the decimal and positional information into a runfile containing instructions for generating a particular bio-tag for a particular well, test tube, container, or location on a container medium. In accordance with some exemplary embodiments, and consistent with a computer executed, substantially automated procedure, the runfile may be embodied in or comprise binary data related to both the unique bio-tags generated and the desired or specified locations for the constituent oligonucleotides thereof.

The mixing apparatus may then execute the instructions contained in the runfile as illustrated at block 418. In accordance with the procedure represented at block 418, a specific and unique bio-tag comprising a selected number and combination of oligonucleotides may be created and deposited in a predetermined container or on a predetermined portion of a container substrate or medium. It will be appreciated that each oligonucleotide, in general, and the specific combination of oligonucleotides, in particular, deposited or provided in block 418 may be selected in accordance with the chemical properties and structural considerations set forth above in detail with specific reference to FIGS. IA and 1B. As indicated at block 419, one or more containers supporting or carrying newly-created bio-tag material may be unloaded from the mixing apparatus and stored, for example, for future use; alternatively, the containers may be used immediately or substantially immediately after bio-tag creation and employed to receive discrete samples as necessary or desired. It will be appreciated that the specific location of each unique bio-tag (i.e., in a particular well of a multi-well plate, for instance, or at a specified coordinate location on an array) may be recorded by the processing component, the mixing apparatus, or both, for future reference and to ensure that a particular sample stored or archived at that location may be properly associated with the bio-tag and later identified substantially as set forth above with particular reference to FIGS. IA and 1B.

FIG. 5 is a simplified flow diagram illustrating the general operation of one embodiment of a method of applying a bio-tag to a sample carrier. As with the method of FIG. 4, the operations depicted at each functional block depicted in FIG. 5 may be executed, controlled, or facilitated by a computer or other processing component encoded with appropriate data and instructions and operating in conjunction with automated or robotic devices.

As indicated at block 511, a prepared container in which bio-tag material is maintained, or a plurality of such containers, may be selectively retrieved as required or desired. In a semi-manual embodiment, an operator may retrieve one or more pre-mixed bio-tag multi-well plates or test tubes, for example, from an inventory; alternatively, retrieval may be entirely automated and executed responsive to control or command signals from the processing component. One or more retrieved bio-tag containers may be loaded into an appropriate apparatus or device, such as a spotting robot or other suitably programmed or dynamically controllable liquid handling machine. As set forth above, while various alternatives exist or may be developed, a Microlab Star liquid handler currently manufactured by and available from Hamilton Company may have particular utility in some applications.

As indicated at block 512, specific bio-tags may be identified (for example, in accordance with a particular well in a multi-well plate or a particular test tube in a rack or other array) and associated data may be recorded for further use; additionally or alternatively, data may be transmitted to control software or other programming scripts executing at the processing component. In accordance with some embodiments, the spotting robot or other automated liquid handler may scan a label or other identifying indicia on the bio-tag containers to facilitate identification thereof; as noted above with reference to FIG. 4, such indicia may be embodied in or comprise a conventional one- or two-dimensional barcode, though other identification strategies may be employed. In some fully automated implementations, various optical barcode readers or machine reading apparatus currently available may be suitable for such identification procedures.

As indicated at block 513, the control software application or computer readable instruction sets executing at the processing component (or under control thereof) may create a data record, for example, or update a data field in a data structure (such as a database, for example) maintained on a storage medium. Created or updated data records may be related specifically to the unique bio-tag intended to be used, and may accordingly be associated therewith when stored in the data structure. Specifically, the processing component may store or update one or more data records to represent the fact that a particular bio-tag identified (at block 512) is to be spotted (i.e., associated, contacted, attached, or otherwise used in conjunction, with a particular sample supporting medium) in subsequent operations.

In addition to storing data as set forth above, and as further indicated at block 513, the processing component may execute instructions operative to ensure that the bio-tag oligonucleotide combination has not been used before; in accordance with this determination, database records for the particular reference system or bio-tag code universe under consideration may be searched or queried for information regarding the identified bio-tag and its associated oligonucleotide combination. If an identified bio-tag has already been used in the reference system or bio-tag universe, an error message may halt the procedure and the processing component may seek operator input, for example, before proceeding; alternatively, a different or alternative bio-tag may be assigned dynamically by the processing component in sophisticated processing embodiments.

Upon confirmation that the bio-tag has not been used previously, data may be transmitted to a label printer (block 514), for example, or to another selected device depending upon system requirements and desired identification protocols. In accordance with the operation depicted at block 514, a label may be embodied in or comprise a one- or two-dimensional barcode or other identifying indicia specifying the intended respective location of each of a plurality of bio-tags in or on a sample carrier (e.g., a multi-well plate or other container, array, or substrate) to be prepared in subsequent operations. In particular, the label may comprise or incorporate coded data associating each bio-tag identified (block 512) and confirmed as available for use (block 513) with a specific and unique well of a multi-well plate to be spotted with a specific and unique bio-tag oligonucleotide combination, for example; alternatively, the coded data may associate each bio-tag with a specific coordinate location on an array or other substrate.

As indicated at block 515, the label created as set forth above may be applied to a sample carrier (i.e., a multi-well plate, array, or other substrate), either manually or automatically, for example, by a robotic apparatus under control of the processing component. In one exemplary embodiment, a sample carrier may comprise a 384 well plate containing FTA filter elements in each well. It will be readily appreciated that different types of plates (e.g., comprising a different number of wells) may also be used, and that different types of sample support media may be employed in addition to, or in lieu of, FTA filter elements. While the following description addresses a multi-well plate for clarity, a sample carrier may also be embodied in or comprise arrays or other substrates having unique, addressable locations disposed thereon or integrated therewith as described above with reference to FIG. 3A.

It will be appreciated that each well in the plate (containing only unspotted and unused filter elements) may not have been unique prior to application of the label, which associates each respective well with a respective unique bio-tag oligonucleotide combination as set forth above. In accordance with such an embodiment, a respective bio-tag may be associated with each respective (otherwise unused) well in the multi-well plate; samples subsequently added to a specific well may be identified in accordance with the bio-tag associated with the well which also contains the sample. In some alternative embodiments in which each well of the multi-well plate already contains a discrete sample, the bio-tag may be associated with the sample as well as the specific location of the well on the plate.

In accordance with the foregoing, an aliquot (such as a 5 μL volume, for example) containing a respective bio-tag solution or compound (i.e., including a unique oligonucleotide combination) may be applied to the filter element, substrate material, or other sample support media contained in each respective well, or to each respective location on a given sample carrier. This application, indicated at block 516, may be performed by any suitable liquid handling apparatus under control of the processing component. In the case where the sample support media has not been contacted with sample material prior to application of the bio-tag solution or compound, each particular location on the sample carrier may now be coded (i.e., associated with an identifying bio-tag) and ready for reception of a discrete sample. As noted above, if the sample carrier already contained discrete samples at identifiable locations, data associated with each respective sample may further be associated with the bio-tag delivered to each respective well.

As indicated at block 517, the spotted sample carrier may be removed from the liquid handler, sealed to prevent contamination in accordance with system requirements or other handling protocols, and delivered, for example, to an inventory or archive facility for storage. As contemplated herein, the operations depicted at block 517 may be executed or facilitated, in whole or in part, by automated handling apparatus or robotic devices operating under control of the processing component such as set forth above. Additionally or alternatively, the spotted sample carrier (appropriately sealed) may be shipped to a third party for additional operations.

The specific arrangement and organization of functional blocks depicted in FIGS. 4 and 5 are not intended to imply a specific order or sequence of operations to the exclusion of other possibilities. For example, the operations illustrated in blocks 511 and 512 may be reversed, or may be performed substantially simultaneously; similarly, the operations depicted at blocks 413 and 414, as well as those depicted at blocks 515 and 516, may be reversed or performed substantially simultaneously. In some embodiments, some operations from both FIGS. 4 and 5 may be selectively combined or omitted in accordance with desired system functionality; for example, the operations depicted at blocks 418 and 516 may be combined such that selected components of the bio-tag solution or compound may be provided directly to a selected portion of a sample carrier as set forth above. Those of skill in the art will appreciate that the specific sequence of operations may be susceptible of various modifications depending, for example, upon myriad factors including, but not limited to, the following: the capabilities and processing bandwidth of the processing component; sophistication and flexibility of the programming instructions executing at the processing component; capabilities and limitations of the liquid handling apparatus and other automated equipment controlled or influenced by the processing component and system software; specific chemistries of the oligonucleotide combinations; desired throughput rates; and other considerations.

Further, in accordance with some exemplary embodiments described above, identifier oligonucleotides may be employed to facilitate bio-tag coding and identification of samples. In cases where each identifier oligonucleotide is immobilized, for instance, at a predetermined or otherwise known location or position on a substrate (e.g., an array), computer executed methods of identifying samples may have particular utility in conjunction with various techniques employed to detect specific hybridization or otherwise to analyze the substrate. For example, identifier oligonucleotides on an array can have a pattern or a configuration such that hybridization results may readily be employed to ascertain which code oligonucleotides are present in an otherwise unknown bio-tagged sample.

Specifically, samples coded with a unique combination of oligonucleotides may be made to contact a substrate (i.e., an array) that includes such identifier oligonucleotides in particular locations and in a predetermined configuration or arrangement, for example. Following contacting with the coded sample, identifier oligonucleotides that specifically hybridize to their complementary code oligonucleotides present in the sample may be detected at particular locations known to correspond to specific identifier oligonucleotides. In the foregoing manner, the code for the bio-tagged sample may be identified or “decoded” based upon which oligonucleotides are present (i.e., those which hybridize with complementary identifier oligonucleotides) and which oligonucleotides are absent (i.e., those which do not hybridize with complementary identifier oligonucleotides). Automated or computer controlled apparatus may be employed to read or otherwise to acquire data from the substrate such that the bio-tagged sample may be identified as set forth above.

Accordingly, a computer executed method of identifying a bio-tagged sample may generally comprise: detecting specific hybridization between a code oligonucleotide and a respective identifier oligonucleotide maintained at a predetermined location on a substrate (such as, for example, an array or bio chip); identifying one or more code oligonucleotides that are present in the bio-tagged sample in accordance with the detecting; comparing the code oligonucleotides present in the bio-tagged sample to data records associating unique oligonucleotide combinations with unique samples; and identifying the bio-tagged sample responsive to the comparing. In some embodiments, the detecting comprises analyzing a hybridization on a substrate having two or more identifier oligonucleotides immobilized at pre-determined positions thereon, wherein the identifier oligonucleotides each have a sequence that is distinct from a sequence present in all other identifier oligonucleotides, and wherein the identifier oligonucleotides are of sufficient number to specifically hybridize to every code oligonucleotide potentially present in the sample. As described in detail above, a substrate having utility in such applications may comprise a plurality of nucleic acid samples immobilized at predetermined positions on the substrate which do not specifically hybridize to code oligonucleotides to the extent that such hybridization prevents code identification.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All publications, patents and other references cited herein are incorporated by reference in their entirety. In case of conflict, the present specification, including definitions, will control.

As used herein, the singular forms “a”, “and,” and “the” include plural referents unless the context clearly indicates otherwise. Thus, for example, reference to “an oligonucleotide or a primer or a sample” includes a plurality of such oligonucleotides, primers and samples, and reference to “an oligonucleotide set” or “a primer set” includes reference to one or more oligonucleotide or primer sets, and so forth.

The invention set forth herein is described with affirmative language. Therefore, even though the invention is generally not expressed herein in terms of what the invention does not include, aspects that are not expressly included in the invention are nevertheless inherently disclosed herein.

A number of embodiments of the invention have been described. Nevertheless, it will be understood that various modifications may be made without departing from the spirit and scope of the invention. Accordingly, the following examples are intended to illustrate but not limit the scope of invention described in the claims.

### EXAMPLES

### Example 1

This example describes an exemplary code using 50, 75 and 100 base oligonucleotides in a single set.

Oligonucleotides comprising the code and corresponding primers were designed by selecting a non-human gene from GENBANK® (NIH genetic sequence database), Arabidopsis thaliana lycopene beta cyclase, accession number U50739, and using the default setting on the Primer 3 program. In order to multiplex the primers in one reaction, the primer pairs were selected from the output of Primer 3 to have a similar melting temperature. To ensure that the sequences selected do not have a significant match to the reported human genes and EST sequences, a Blast comparison was performed against GENBANK® (NIH genetic sequence database) non-redundant (nr) database. Oligonucleotide and primer sequences were as follows:

(SEQ ID NOs: 1-3, respectively)

50 bp oligonucleotide, PCR primer #1
5′ TCCATCTCCATGAAGCTACT 3′
50 bp oligonucleotide, PCR primer #2
5′ ATGAACGAAGACCACAAAAC 3′
50 bp oligonucleotide
5′ CCATCTCCATGAAGCTACTGCTTCTGGGTAAGTTTTGTGGTCTTCGT
TCAT 3′

(SEQ ID NOs: 4-6, respectively)

75 bp oligonucleotide, PCR primer #1
5′ GTGTCAAGAAGGATTTGAGC 3′
75 bp oligonucleotide, PCR primer #2
5′ TTTCTGAAGCATTTTGGATT 3′
75 bp oligonucleotide
5′ GTGTCAAGAAGGATTTGAGCCGGCCTTATGGGAGAGTTAACCGGAAA
CAGCTCAAATCCAAAATGCTTCAGAAA 3′

(SEQ ID NOs: 7-9, respectively)

100 bp oligonucleotide, PCR primer #1
5′ TCTGAAGCTGGACTCTCTGT 3′
100 bp oligonucleotide, PCR primer #2
5′ AATCCATAGCCTCAAACTCA 3′
100 bp oligonucleotide
5′ TCTGAAGCTGGACTCTCTGTTTGTTCCATTGATCCTTCTCCTAAGCT
CATATGGCCTAACAATTATGGAGTTTGGGTTGATGAGTTTGAGGCTATGG
ATT 3′

The oligonucleotides were applied to the media in solution. A solution is made up of the desired combination of oligonucleotides at a concentration of 0.1 uM each. Three microliters of the solution is then applied to the media (FTA or Iso-Code) and allowed to dry, either at room temperature or in a desiccator at room temperature.

PCR was performed on different mixtures of the 50 bp, 75 bp, and 100 by oligonucleotides. The PCR reaction mixture contained: 16 mM (NH₄)₂SO₄, 67 mM Tris-HCl (pH 8.8 at 25C), 0.01% Tween 20, 1.5 mM MgCl), 200 μM each dNTP (Bioline, Randolph, Mass.), 0.1 μM of each primer (all three primer pairs were present in each reaction), and 2 units of Biolase (Bioline, Randolph, Mass.). The PCR cycling conditions were as follows: 93° C. for 2 minutes, 55° C. for 1 minute, 72° C. for 2 minutes, followed by 25 cycles of 93° C. for 30 seconds, 55° C. for 30 seconds, 72° C. for 45 seconds.

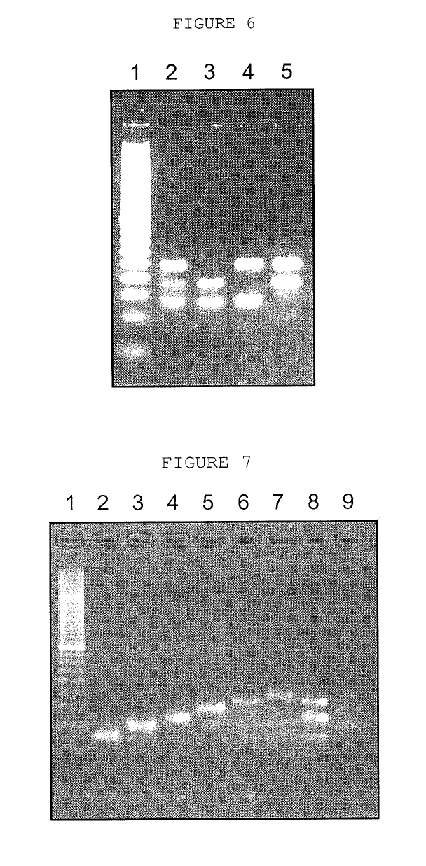

The PCR products were analyzed on a 3% agarose gel in 1×TBE, run for 1 hour at 150V. An image of the resulting gel is shown in FIG. 6. Lane 1 is 20 by ladder by Apex (DocFrugal Scientific, La Jolla, Calif.); lane 2 contains 0.1 μM of each of the three oligonucleotides; lane 3 contains 0.1 μM of the 50 by and 75 by oligonucleotides; lane 4 contains 0.1 μM of the 50 by and 100 by oligonucleotides; and lane 5 contains 0.1 μM of the 75 by and 100 by oligonucleotides.

An oligonucleotide set having 50, 60, 70, 80, 90, and 100 base oligonucleotides was also designed. Oligonucleotide and primer sequences were as follows (the 50 and 100 base oligonucleotides and corresponding primers were as described above):

(SEQ ID NOs: 10-12, respectively)

60 bp oligonucleotide, PCR primer #1
5′ GGCTATTGTTGGTGGTGGTC 3′
60 bp oligonucleotide, PCR primer #2
5′ TCCAGCTTCAGAAACCTGCT 3′
60 bp oligonucleotide
5′ GCTATTGTTGGTGGTGGTCCTGCTGGTTTAGCCGTGGCTCAGCAGGT
TTCTGAAGCTGGA 3′

(SEQ ID NOs: 13-15, respectively)

70 bp oligonucleotide, PCR primer #1
5′ CAAACTCCACTGTGGTCTGC 3′
70 bp oligonucleotide, PCR primer #2
5′ AACCCAGTGGCATCAAGAAC 3′
70 bp oligonucleotide
5′ AAACTCCACTGTGGTCTGCAGTGACGGTGTAAAGATTCAGGCTTCCG
TGGTTCTTGATGCCACTGGGTT 3′

(SEQ ID NOs: 16-18, respectively)

80 bp oligonucleotide, PCR primer #1
5′ TGGTGTTCATGGATTGGAGA 3′
80 bp oligonucleotide, PCR primer #2
5′ GAACGTTGGGATCTTGCTGT 3′
80 bp oligonucleotide
5′ TGGTGTTCATGGATTGGAGAGACAAACATCTGGACTCATATCCTGAG
CTGAAGAACGGAACAGCAAGATCCCAACGTTC

(SEQ ID NOs: 19-21, respectively)

90 bp oligonucleotide, PCR primer #1
5′ GGGGATCAATGTGAAGAGGA 3′
90 bp oligonucleotide, PCR primer #2
5′ CCACAACCCGTTGAGGTAAG 3′
90 bp oligonucleotide
5′ GGGGATCAATGTGAAGAGGATTGAGGAAGACGAGCGTTGTGTGATCC
CGATGGGCGGTCCTTTACCAGTCTTACCTCAACGGGTTGTGG 3′

This additional set of oligonucleotides was analyzed by PCR as described above and the results are shown in FIG. 7. Lane 1 is the 20 by ladder by Apex (DocFrugal Scientific, La Jolla, Calif.); lane 2 contains 0.1 μM of a 50 by oligonucleotide; lane 3 contains 0.1 μM of a 60 by oligonucleotide; lane 4 contains 0.1 μM of a 70 by oligonucleotide; lane 5 contains 0.1 μM of a 80 by oligonucleotide; lane 6 contains 0.1 μM of a 90 by oligonucleotide; lane 7 contains 0.1 μM of a 100 by oligonucleotide; lane 8 contains 0.1 μM of each of the 50, 70, and 90 by oligonucleotides; and lane 9 contains 0.1 μM of each of the 60, 80, and 100 by oligonucleotides.

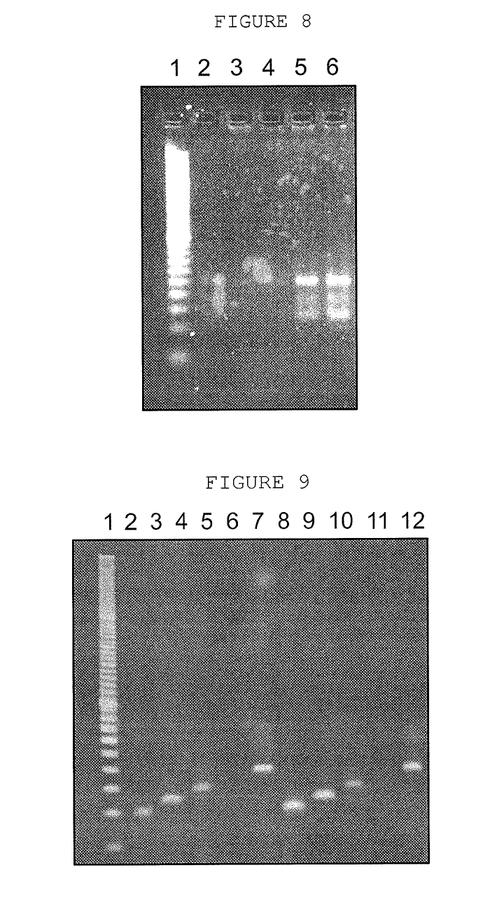

The 50, 75, 100 base oligonucleotide set was also analyzed by PCR after being mixed with human blood on FTA™ paper and Iso-Code™ paper, as shown in FIG. 8. Lane 1 is the 20 by ladder by Apex (DocFrugal Scientific, La Jolla, Calif.). Lanes 2-6 are 10 μL of a PCR reaction containing the three primer pairs. Lane 2 is a no template control. The templates for the remaining lanes are as follows: lane 3 is a 3 mm circle of FTA™ paper that contains human blood; lane 4 is a 3 mm circle of Iso-Code™ paper that contains human blood; lane 5 is a 3 mm circle of FTA™ paper that contains both human blood and 50, 75, and 100 by oligonucleotides; and lane 6 is a 3 mm circle of FTA™ paper that contains both human blood and 50, 75, and 100 by oligonucleotides.

### Example 2

This example describes an exemplary code using 50, 60, 70, 80, 90 and 100 base oligonucleotides in two sets. Set #2 was designed from the Arabidopsis thaliana At3g59020 mRNA sequence, while set #3 was designed from the Arabidopsis thaliana At5g18620 mRNA sequence. Oligonucleotide and primer sequences were as follows:

Set #2
At3g59020 mRNA sequence

(SEQ ID NOs: 22-24, respectively)

50 bp oligonucleotide, PCR primer #1
5′ GCACCCATTCACCGAGTAGT 3′
50 bp oligonucleotide, PCR primer #2
5′ ATGTTCAACAGGTGGGGAAA 3′
50 bp oligonucleotide
5′ GCACCCATTCACCGAGTAGTCGAGGAGACTTTTCCCCACCTGTTGAA
CAT 3′

(SEQ ID NOs: 25-27, respectively)

60 bp oligonucleotide, PCR primer #1
5′ CAGTTTTTGCTTTGCGTTCA 3′
60 bp oligonucleotide, PCR primer #2
5′ CTGGGCGGATTTCATCTAAA 3′
60 bp oligonucleotide
5′ CAGTTTTTGCTTTGCGTTCATTTATTGAAGCCTGCAAAGATTTAGAT
GAAATCCGCCCAG 3′

(SEQ ID NOs: 28-30, respectively)

70 bp oligonucleotide, PCR primer #1
5′ TCAAGTGCCTTCTGGTTGAA 3′
70 bp oligonucleotide, PCR primer #2
5′ AGTATGCCAAGTGCCAAAGG 3′
70 bp oligonucleotide
5′ TCAAGTGCCTTCTGGTTGAAGTGGTTGCAAATGCCTTTTACTACAAT
ACCCCTTTGGCACTTGGCATACT 3′

(SEQ ID NOs: 31-33, respectively)

80 bp oligonucleotide, PCR primer #1
5′ TCGACACTGACAACGGTGAT 3′
80 bp oligonucleotide, PCR primer #2
5′ GGTACTGATGGCACGGAGAC 3′
80 bp oligonucleotide,
5′ TCGACACTGACAACGGTGATGATGAAACTGATGATGCTGGTGCATTG
GCTGCAGTGGGATGTCTCCGTGCCATCAGTACC 3′

(SEQ ID NOs: 34-36, respectively)

90 bp oligonucleotide, PCR primer #1
5′ CGAGTCTCGTCGATTTCCTC 3′
90 bp oligonucleotide, PCR primer #2
5′ TTAAAGCGAGGCTAGGCAGA 3′
90 bp oligonucleotide
5′ CGAGTCTCGTCGATTTCCTCCGGGAGGAGACTTGAAATTCGTGACTT
TCCGATTGTGAATTCCCCGATGGATCTGCCTAGCCTCGCTTTAA 3′

(SEQ ID NOs: 37-39, respectively)

100 bp oligonucleotide, PCR primer #1
5′ GTCTCCGTGCCATCAGTACC 3′
100 bp oligonucleotide, PCR primer #2
5′ AGCATTTTCCGCATTATTGG 3′
100 bp oligonucleotide
5′ GTCTCCGTGCCATCAGTACCATTCTTGAATCTATCAGTGTCTCCCTC
ATCTTTATGGTCAGATTGAACCACAGTTACTGCCAATAATGCGGAAAATG
CT 3′
Set #3
At5gl 8620 mRNA sequence

(SEQ ID NOs: 40-42, respectively)

50 bp oligonucleotide, PCR primer #1
5′ TGTCTCTGACGACGAGGTTG 3′
50 bp oligonucleotide, PCR primer #2
5′ CGTCCTCTTCAGCGTCATCT 3′
50 bp oligonucleotide
5′ TGTCTCTGACGACGAGGTTGTCCCCGTAGAAGATGACGCTGAAGAGG
ACG 3′

(SEQ ID NOs: 43-45, respectively)

60 bp oligonucleotide, PCR primer #1
5′ GGAGAACGCAAACGTCTGTT 3
60 bp oligonucleotide, PCR primer #2
5′ AAGGGTGATTGCAGCATTTC 3′
60 bp oligonucleotide
5′ GGAGAACGCAAACGTCTGTTGAACATAGCAATGCATTGCGGAAATGC
TGCAATCACCCT 3′

(SEQ ID NOs: 46-48, respectively)

70 bp oligonucleotide, PCR primer #1
5′ AGGAACCCTCGATTCGATCT 3′
70 bp oligonucleotide, PCR primer #2
5′ TCGAAGCTCTAGCCATCGAC 3′
70 bp oligonucleotide
5′ AGGACCCTCGATTCGATCTCTCAGACGAAATCAGGATTCGTAGAGGC
GCGTCGATGGCTAGAGCTTCGA 3′

(SEQ ID NOs: 49-51, respectively)

80 bp oligonucleotide, PCR primer #1
5′ CCCTCGATTCGATCTCTCAG 3′
80 bp oligonucleotide, PCR primer #2
5′ GAAGAAACTTCCCGCTTCG 3′
80 bp oligonucleotide
5′ CCTCGATTCGATCTCTCAGACGAAATCAGGATTCGTAGAGGCGCGTC
GATGGCTAGAGCTCGAAGCGGGAAGTTTCTTC 3′

(SEQ ID NOs: 52-54, respectively)

90 bp oligonucleotide, PCR primer #1
5′ CAGCAAACGTGAGAAGGCTA 3′
90 bp oligonucleotide, PCR primer #2
5′ TGGAAGCATTTTGGGAGTCT 3′
90 bp oligonucleotide
5′ CAGCAAACGTGAGAAGGCTAGACTCAAAGAAATGCAGAAGATGAAGA
AGCAGAAAATTCAGCAAATCTTAGACTCCCAAAATGCTTCCA 3′

(SEQ ID NOs: 55-57, respectively)

100 bp oligonucleotide, PCR primer #1
5′ GCCGATTTTGTCCTGTCCT 3′
100 bp oligonucleotide, PCR primer #2
5′ ATGTCGAATTTCCCTGCAAC 3′
100 bp oligonucleotide
5′ GCCGATTTTGTCCTGTCCTGCGTGCTGTGAAATTTCTCGGTAATCCC
GAGGAAAGAAGACATATTCGTGAAGAACTGCTAGTTGCAGGGAAATTCGA
CAT 3′

The oligonucleotides of Set #2 and Set #3 were amplified by PCR. With each set of primers being separated by 10 bases, a 6% polyacrylamide gel was employed (Invitrogen, Carlsbad). The PCR reaction conditions and the amount of oligonucleotide were as described above. The corresponding PCR primer concentration was reduced from 0.1 uM per reaction to 0.05 uM. The results for Set #2 are shown in FIG. 9. Lane 1 is the 20 by ladder by Apex (DocFrugal Scientific, La Jolla, Calif.). Lanes 2-7 each contain all 5 primer pairs from Set #2 but only 1 of the oligonucleotides from the set. Lanes 8-12 each contain only 1 set of primer pairs from Set #2, but all 5 of the Set #2 oligonucleotides.

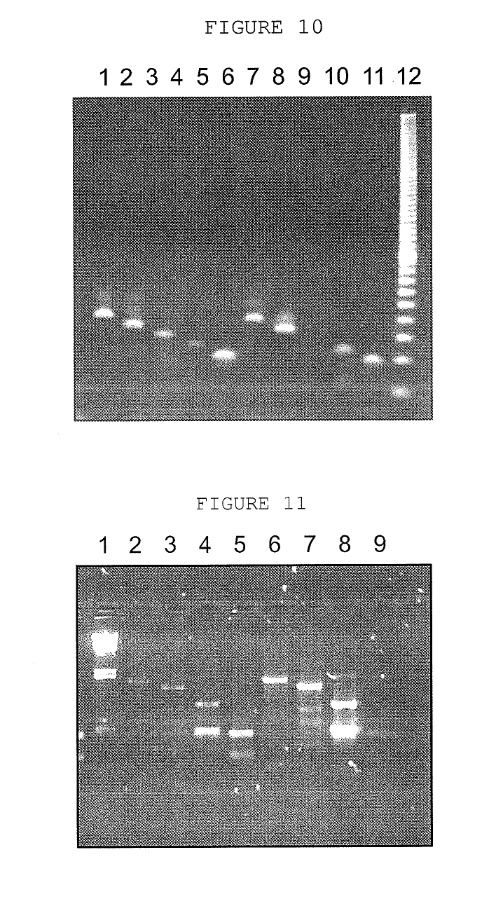

Likewise, the results for Set #3 are shown in FIG. 10. Lane 1 is the 20 by ladder by Apex (DocFrugal Scientific, La Jolla, Calif.). Lanes 7-11 each contain all 5 primer pairs from Set #3 but only 1 of the oligonucleotides from the set. Lanes 1-6 each contain only 1 set of primer pairs from Set #3, but all 5 of the Set #3 oligonucleotides.

### Example 3

Enhancement of PCR with the Presence of the Bio-Tag.

The addition of oligonucleotides to the matrix prior to the addition of blood enhances the amount of PCR yield. The oligonucleotide code is applied to the matrix and allowed to dry completely prior to the addition of blood. FIG. 11 shows the results of β-actin amplification from blood samples applied to matrix alone or matrix that had oligonucleotides pre-applied. PCR was performed and analyzed as described above, using the β-actin primers described below. The PCR cycling conditions were: 93° C. for 2 minutes, 55° C. for 1 minute, 72° C. for 2 minutes, followed by 25 cycles of 93° C. for 45 seconds, 55° C. for 45 seconds, 72° C. for 2 minutes. Lane 1 is a HindIII ladder (New England Biolabs, MD.). Lanes 2 and 6 contain 10 μM of each of the full β-actin primers (2 kb). Lanes 3 and 7 contain 10 μM of each of the 1.5 kb β-actin primers. Lanes 4 and 8 contain 10 μM of each of the 1.0 kb β-actin primers. Lanes 5 and 9 each contain 10 μM of each of the 500 by β-actin primers. Lanes 2-4 do not contain any oligonucleotides; and lanes 5-9 contain 0.1 μM of the 50, 75, and 100 bp oligonucleotides.

Beta Actin Primers

| All reactions used the same primer #1: | |
|---|---|
| 5′ agcacagagcctcgccttt 3′ | (SEQ ID NO: 58) |

(SEQ ID NOs: 59-62, respectively)

| | |
|---|---|
| 2 kb primer #2 | 5′ GGTGTGCACTTTTATTCAACTGG 3′ |
| 1.5 kb primer #2 | 5′ AGAGAAGTGGGGTGGCTTTT 3′ |
| 1.0 kb primer #2 | 5′ AGGGCAGTGATCTCCTTCTG 3′ |
| 0.5 kb primer #2 | 5′ AGAGGCGTACAGGGATAGCA 3′ |

### Example 4

This example describes particular inherent properties of certain embodiments of the invention.

Inherent in the invention is the difficulty with which counterfeiters could identify and, therefore, reproduce the code. When using multiple (e.g., two or more) sets of oligonucleotides in which there is at least one oligonucleotide from the two sets having an identical length, it is impossible to reproduce the specific banding pattern created by the code without knowing the primers that specifically hybridize to the oligonucleotides. For example, although there are technologies that could provide the requisite sensitivity and resolution needed to visualize the bio-code on a gel without amplifying the oligonucleotides, this data would be worthless since there are at least two oligonucleotides having the same size in the code, which could not be size-differentiated in one dimension. Furthermore, although random primed PCR could be attempted to clone and sequence the oligonucleotides comprising the code, this would simply generate a ladder up to the largest oligonucleotide present in the particular mixture, not the correct code pattern. When the oligonucleotides comprising the code are single strand, there is no practical way to clone single strand sequences into vectors to try and duplicate the combination of oligonucleotides comprising the code. Thus, in contrast to computer based encoding, electronic based authenticating markers, or watermarks which can eventually be duplicated with ever advancing computing capabilities, the code is not easily identified and, therefore, cannot be reproduced without knowing the sequences of the primers.

### Example 5

This example describes various non-limiting specific applications of the bio-code.

Forensic Chain of Evidence Assurance: Forensic samples such as blood and body fluids or tissues that are collected at the scene of a crime or from a suspect using evidence collection kits based upon paper, or treated papers such as FTA™ (Whatman) or IsoCode™ (Schleicher and Schuell). A bar-coded card is used to write down date, time, location, collector and other relevant information so that it stays with the collection card. When analysis of the sample on the collection card (e.g., nucleic acid) is desired, a 1 or 2 mm punch is taken from the portion of the collection card with the forensic sample, e.g., where the sample was collected. The nucleic acid is subsequently identified using commercially available human ID kits such as are provided by Promega and other commercial sources. These kits provide a buffer for washing the cellular debris and proteins from the nucleic acid purifying it for subsequent multiplex PCR for human identification.

A series of 25 different oligonucleotides chosen to avoid sequence commonality with the human genome are used to generate a unique bio-barcode similar to the exemplary illustration (FIGS. 1 and 2) described herein. The unique code at a concentration set to provide a total of 5 ng/cm² is added to the card and allowed to dry. When the forensic sample is analyzed, for example, to ID the human based upon the DNA present, five additional PCR reactions are included to develop the bio-barcode. When the PCR reactions are fractionated via gel electrophoresis, the additional five lanes appear as barcode which is directly linked with the human ID information and with the sample on the original collection card. This method is advantageous because the means to develop the code are the same as that used to analyze the genetic material of the sample. Accordingly, the code directly links the ID of the individual to the information on the card used to collect the sample. Even though a punch might be initially mis-identified by a laboratory technician, all ambiguity is removed as soon as the bar-code of the punched section is developed. An additional feature is that a scan or digital image of the gel with both the nucleic acid sample and the bar-code will contain not only the identification information for the individual but also the direct link to the evidence, ensuring a rigid chain of custody to the location where the forensic sample was collected.

High Value Documents: Paper documents such as commercial paper, bonds, stocks, money, etc. can be ensured to be authentic by implanting upon the paper and valid copies, a unique combination of oligonucleotides providing a barcode. If the validity of the document is in question, a sample of the paper is taken and the code developed, for example, via PCR amplification and subsequent gel electrophoresis. If the barcode is absent or does not match the expected code, then the item is counterfeit. Similarly, by the attachment of a small swatch of paper or fabric to any high value item, authenticity of the item can be ensured.

Again, the use of 25 primer pairs that specifically hybridize to 25 oligonucleotides in a binary (present or not present) code can be use to uniquely identify over 34 million different documents. By using 30 oligonucleotides and six lanes of 5 primer pairs each, the system can be used to uniquely identify over one billion different documents. Cost per document can be as low as a few cents or less if the code material is placed in a specific location on the document such as part of the letterhead or a designated area of the print information on the document. A wax or other seal (organic or inorganic) could also be placed over the code material to protect against possible loss or degradation.

Sample Storage/Archiving: In an automated sample store (i.e., archive), study assembly consists of selecting multiple samples from the archive and assembling them into a daughter plate (typically a lab microplate consists of 100 to 1000 wells, each capable of containing a distinct sample). Clinical samples of this type are typically valued at about $100 each, so mistakes in sample assembly or a mishap during or after sample retrieval resulting in the samples being scrambled would be extremely costly. Although some of this risk can be avoided through careful package and process design (i.e., sample storage, retrieval and tracking), a code for each sample when the sample is introduced into the archive so that the sample can be distinguished from others and traced back to their original source provides additional protection.

One can code every sample that enters the sample store. However, it is not necessary to code every sample. For example, samples can be coded upon retrieval from the store, which is more economical since fewer codes are required and because the coding expense is incurred only for those samples that leave the archive rather than for every sample that enters the archive. In any event, the oligonucleotide code can be added to or mixed with every sample introduced into the store or only those samples that leave the store.

### Example 6

This example describes an exemplary application of a microarray that includes identifier oligonucleotides, which are used to develop the code present in a sample.

Illumina Gene Expression Profiling

A sample having a code is applied to an array in which a portion of the array has identifier oligonucleotides that can be used to specifically hybridize to all oligonucleotides of the code. As an example, an Illumina array could have part of one row or column of the array with identifier oligonucleotides, each at pre-determined positions, to develop the sample code. Alternatively, the array could be set up to use a 5×6 section (30 identifier oligonucleotides) to present the same image as the gel electrophoresis scans (2-D bar-code, see FIG. 1). Since the Illumina system is based upon 50mers, the identifier oligonucleotides can be easily included in the array.

An Illumina Sentrix® Array matrix has 96 array clusters. Each array cluster in each multi-sample platform can query over 700 genes, with two 50-mer probes per gene. The array matrix can be pre-prepared with customer-specified oligonucleotides to identify specific DNA sequences, including the oligonucleotides of the code. DNA samples greater than 50 ng can be directly applied to the array to detect specific hybridization between the sample DNA and the oligonucleotides of the array, and the code oligonucleotides and the identifier oligonucleotides. A positive hybridization signal for a code oligonucleotide would represent a 1 and a lack of response a 0, providing a binary number identifying the code and, therefore, the sample. Where the sample was from a GenVault plate, the binary number would also represent the plate type, plate number and a check code to verify a good read.

More particularly, a sample of nucleic acid containing a bio-tag from an appropriate source, such as a GenVault DNA storage plate, is eluted as purified dsDNA. After preparation, such as concentration of the sample, typically the amount of eluted DNA will be less than 50 ng. The DNA is subsequently amplified using a highly multiplexed PCR process to provide a sufficient quantity of nucleic acid for hybridization and detection. The multiplex PCR includes primer pairs that specifically hybridize to the code oligonucleotides, as well as other DNA sequences of interest. Following PCR, the mixture of amplified sample nucleic acid and code oligonucleotides is cleaned up to remove excess primers and, if necessary, provide a suitable buffer for array hybridization. The amplified mixture is contacted to the array under conditions allowing specific hybridization to occur. Upon development of the array, both the identity of the sample via the unique combination of oligonucleotides in the code and the presence, or absence, of target sequences of interest become readily apparent. A digital record of the developed array and sample identification, which resides on the array, provides a direct link between the identity of the sample and the array data for the sample.

As set forth above, a bio-tag may generally be associated with information regarding the sample identity, source, patient data, etc. By including the bio-tag in the sample itself (i.e., by co-locating the unique combination of oligonucleotides with the sample material), an internal sample identification check is possible prior to, at the time of the “read” process, and later in reviewing a record of array data. Additionally, by reading the bio-tag code associated with the sample, as well as a container barcode or other indicia (for example, associated with a particular sample carrier such as a multi-well plate) into a computer or other processing component and associating the bio-tag with the container or sample carrier code, an irrevocable link between sample identification, patient data, and any other information desired allows any particular sample to be tracked through data linking that sample with a container or sample carrier having a unique code. In some embodiments, for example, a container code such as mentioned above may be represented as a decimal version of the binary bio-tag code associated with a sample, and may be used to link a bio-tagged sample with a particular sample carrier or location thereon for traceability or tracking purposes. Specifically, container information and other data may be encoded in a label bearing a barcode or other indicia substantially as set forth above; such a label may be affixed to the sample carrier, and may also include additional information, for instance, identifying the type of sample carrier, the number of samples remaining, and so forth. Such data may be employed by software or automated apparatus operative to retrieve or otherwise to handle sample carriers and sample material extracted or removed therefrom.

Additionally, a check code may readily be implemented to verify a good read on the bio-tag code for a particular sample. By using, for example, part of an Illumina array for oligonucleotide identifiers of the code, a code may be generated for patient A nucleic acid, a different code may be generated for patient B nucleic acid, and so forth. In the foregoing manner, confirmation may be made of the correctness of the read. In that regard, if a bio-tag read indicates that a sample is from patient A, but the check code indicates otherwise, an error in the read may be the cause for such a discrepancy. Alternatively, where the check code and the bio-tag code are consistent, an accurate read can be confirmed. A check code in this context may be embodied in or comprise a set oligonucleotides (e.g., approximately five oligonucleotides), the presence or absence of which may be a function of the other oligonucleotides that make up the bio-tag. In some embodiments, the bio-tag code and the check code may be combined, for example, or otherwise integrated to serve as a unique identifier for a particular sample.

By way of example, and not by way of limitation, a 5-bit CRC (Cycle Redundancy Check) algorithm may be implemented to determine the check code; CRC's are generally known in the art, and have utility in check code applications for binary data transmission (i.e., sending electronic data). A 5-bit CRC may readily identify false negatives/positives in resolving the code, and are sufficient to identify lane swaps or errors in reading the data out of order; this may be appropriate in instances where a configuration containing 5-bit lanes such as indicated in FIG. 2A is employed. Alternatively, more processor intensive CRC's may be implemented in accordance with generally known principles and in accordance with system hardware configurations and desired system performance.

A personalized code may be employed to identify a given sample with even more particularity or granularity. For example, a personalized or institutional code may be embodied in or comprise any of various other suitable algorithms or identifiers that a particular institution desired to use; in some embodiments, such a personalized code may be used in addition to, or in lieu of, the CRC check code described above. In the foregoing manner, hospitals, clinics, research and other laboratories, or any other entity may use a field for a “personalized code” unique to the particular institution. This would function as an internal check on the accuracy of the identification of the sample as well as a check on “wayward” samples.

Affymetrix GeneChip® Arrays

GeneChip® arrays contain hundreds of thousands of oligonucleotide probes at extremely high densities. The probes allow discrimination between specific and background signals, and between closely related target sequences. GeneChip® arrays, which have been used for a wide variety of DNA and mRNA analyses, can include identifier olignucleotides in accordance with the invention in order to identify a code present in a sample.

A sample of purified dsDNA, containing an oligonucleotide sequence code is prepared via a modified Affymetrix protocol, and applied to the GeneChip®. Optionally, PCR of the sample using biotinylated nucleic acids can be performed to increase the amount of DNA or the amount of code oligonucleotides present in the sample. As in the Illumina example, the coded sample is applied to the GeneChip®. The absence or presence of a code oligonucleotide in the sample is determined by the absence or presence of a detectable signal at the specific position on the GeneChip® having the identifier olignucleotide that specifically hybridizes to the code oligonucleotide. Simultaneous conventional nucleic acid hybridization between the sample and the oligonucleotide probes of the GeneChip® array detects the presence of selected SNPs or heterozygous sequence changes in the dsDNA sample.

What is claimed:

1. A storage package comprising:
a container containing a combination of two or more oligonucleotides from a predetermined pool of oligonucleotides; and
an identifying indicia attached to said container,
wherein the oligonucleotides of said pool are different from each other, wherein the combination of oligonucleotides represents the presence and absence of oligonucleotides from said pool and such representation constitutes a code, and wherein said identifying indicia identifies the code represented by said combination of oligonucleotides.

2. The storage package of claim 1, wherein said combination comprises five or more oligonucleotides from said pool.

3. The storage package of claim 1, wherein the oligonucleotides from said combination belong to two or more sets.

4. The storage package of claim 1, wherein the oligonucleotides of said combination have different length from each other.

5. The storage package of claim 3, wherein oligonucleotides within a set have different length from each other.

6. The storage package of claim 1, wherein each oligonucleotide of said combination is from about 8 to 5000 nucleotides long.

7. The storage package of claim 1, wherein each oligonucleotide of said combination comprises a pair of primer hybridizing sequences.

8. The storage package of claim 7, wherein said primer hybridizing sequences are located at the ends of each oligonucleotide of said combination.

9. The storage package of claim 7, wherein the oligonucleotides from said combination belong to two or more sets, wherein each oligonucleotide in the same set has the same pair of primer hybridizing sequences, and wherein the pair of primer hybridizing sequences is different for each set.

10. The storage package of claim 1, further comprising one or more pairs of primers, wherein said one or more pairs of primers specifically hybridize to each oligonucleotide of said combination.

11. The storage package of claim 1, further comprising one or more pairs of primers, wherein said one or more pairs of primers specifically hybridize to each oligonucleotide of said pool.

12. The storage package of claim 3, further comprising two or more pairs of primers, wherein each pair of primers specifically hybridizes to each oligonucleotide in a set.

13. The storage package of claim 1, comprising a plurality of containers or a multi-well plate as said container.

14. The storage package of claim 1, wherein said container comprises a substrate for biological molecule storage, and wherein said substrate contains said combination of oligonucleotides.

15. The storage package of claim 14, wherein said substrate is a dry solid medium.

16. The storage package of claim 15, wherein said dry solid medium comprises cellulose, polyester, plastic, or a combination thereof.

17. The storage package of claim 14, wherein said substrate is an absorbent foam.

18. The storage package of claim 1, wherein said identifying indicia is a barcode.

19. The storage package of claim 1, comprising a combination of five or more oligonucleotides from said pool,
wherein said combination of oligonucleotides has the following formula: A1-A2-A3-A4-A5-B1-B2-B3-B4-B5-C1-C2-C3 -C4-C5-D1-D2-D3-D4-D5-E1-E2-E3-E4-E5,
wherein each letter with its number designation in the formula represents an oligonucleotide which is either present or absent from the combination, wherein oligonucleotides represented by the same letter belong to a set, and wherein oligonucleotides in the same set have a common set characteristic.

20. The storage package of claim 1, comprising a combination of five or more oligonucleotides from said pool,
wherein said combination of oligonucleotides has the following formula: A1-A2-A3-A4-B1-B2-B3-B4-C1-C2-C3-C4-D1-D2-D3-D4-E1-E2-E3-E4-F1-F2-F3-F4,
wherein each letter with its number designation in the formula represents an oligonucleotide which is either present or absent from the combination, wherein oligonucleotides represented by the same letter belong to a set, and wherein oligonucleotides in the same set have a common set characteristic.

21. The storage package of claim 1, comprising a combination of five or more oligonucleotides from said pool,
wherein said combination of oligonucleotides has the following formula: A1-A2-A3-A4-A5-B1-B2-B3-B4-B5-C1-C2-C3-C4-C5-D1-D2-D3-D4-D5-E1-E2-E3-E4-E5-F1-F2-F3-F4-F5,
wherein each letter with its number designation in the formula represents an oligonucleotide which is either present or absent from the combination, wherein oligonucleotides represented by the same letter belong to set, and wherein oligonucleotides in the same set have a common set characteristic.

22. The storage package of claim 19, wherein each oligonucleotide in the same set has the same pair of primer hybridizing sequences, and wherein the pair of primer hybridizing sequences is different for each set.

23. The storage package of claim 22, further comprising five or more pairs of primers, wherein each pair of primers specifically hybridizes to each oligonucleotide in the same set.

24. The storage package of claim 19, further comprising five or more pairs of primers, wherein said pairs of primers specifically hybridize to each oligonucleotide of said combination.

25. The storage package of claim 19, further comprising five or more pairs of primers, wherein said pairs of primers specifically hybridize to each oligonucleotide of said pool.

26. The storage package of claim 1, further comprising a biological sample.

27. The storage package of claim 19, further comprising a biological sample.

28. The storage package of claim 26, wherein each oligonucleotide of said combination is incapable of specifically hybridizing to said sample.

29. The storage package of claim 27, wherein each oligonucleotide of said combination is incapable of specifically hybridizing to said sample.

30. An archive of biological samples, wherein each sample is stored in a container of claim 1.

31. An archive of biological samples, wherein each sample is stored in a container of claim 19.

32. A method for coding a sample comprising adding a sample to a container of claim 1.

33. A method for coding a sample comprising adding a sample to a container of claim 19.

34. A method for coding a plurality of discrete samples comprising adding each sample of said plurality to a container of claim 1.

35. A method for coding a plurality of discrete samples comprising adding each sample of said plurality to a container of claim 19.

36. A method of decoding a sample contained in a container of claim 1 comprising detecting the presence and absence of one or more oligonucleotides of said pool in said container, wherein a collective result of the presence and absence of one or more oligonucleotides of said pool in said container is indicative of a code associated with the sample.

37. A method of decoding a sample contained in a container of claim 19 comprising detecting the presence and absence of at least five oligonucleotides of said pool in said container, wherein a collective result of the presence and absence of at least five oligonucleotides of said pool in said container is indicative of a code associated with the sample.

38. The method of claim 36, comprising detecting the presence and absence of each oligonucleotide of said pool in said container.

39. The method of claim 37, comprising detecting the presence and absence of each oligonucleotide of said pool in said container.

40. A kit comprising:
a container containing a combination of two or more oligonucleotides from a predetermined pool of oligonucleotides; and
an identifying indicia,
wherein the oligonucleotides of said pool are different from each other, and wherein the combination of oligonucleotides represents the presence and absence of oligonucleotides from said pool and such representation constitutes a code.

41. The kit of claim 40, further comprising one or more pairs of primers, wherein each oligonucleotide of said combination has a pair of primer hybridization sequences, and wherein said one or more pairs of primers specifically hybridize to each oligonucleotide from said combination.

42. The kit of claim 40, wherein said identifying indicia is attached to said container.

43. The kit of claim 40, wherein said container comprises a substrate for biological molecule storage.

44. A kit comprising at least five pairs of primers, wherein said pairs of primers specifically hybridize to each oligonucleotide from a pool of oligonucleotides represented by the formula A1-A2-A3-A4-A5-B1-B2-B3-B4-B5-C1-C2-C3-C4-C5-D1-D2-D3-D4-D5-E1-E2-E3-E4-E5, wherein each letter with its number designation in the formula represents an oligonucleotide, wherein oligonucleotides represented by the same letter belong to a set, and wherein oligonucleotides in the same set have a common primer hybridization sequence.

45. The kit of claim 44, wherein said pairs of primers specifically hybridize to the primer hybridization sequences of each set.

46. The kit of claim 44, further comprising an instruction connecting said pairs of primers to said pool of oligonucleotides.


SEQUENCE LISTING (62 sequences)

SEQ ID NO: 1 - DNA, 20 bp, Arabidopsis thaliana
tccatctccatgaagctact

SEQ ID NO: 2 - DNA, 20 bp, Arabidopsis thaliana
atgaacgaagaccacaaaac

SEQ ID NO: 3 - DNA, 51 bp, Arabidopsis thaliana
ccatctccatgaagctactgcttctgggtaagttttgtggtcttcgttcat

SEQ ID NO: 4 - DNA, 20 bp, Arabidopsis thaliana
gtgtcaagaaggatttgagc

SEQ ID NO: 5 - DNA, 20 bp, Arabidopsis thaliana
tttctgaagcattttggatt

SEQ ID NO: 6 - DNA, 74 bp, Arabidopsis thaliana
gtgtcaagaaggatttgagccggccttatgggagagttaaccggaaacagctcaaatccaaaatgcttcagaaa

SEQ ID NO: 7 - DNA, 20 bp, Arabidopsis thaliana
tctgaagctggactctctgt

SEQ ID NO: 8 - DNA, 20 bp, Arabidopsis thaliana
aatccatagcctcaaactca

SEQ ID NO: 9 - DNA, 100 bp, Arabidopsis thaliana
tctgaagctggactctctgtttgttccattgatccttctcctaagctcatatggcctaacaattatggagtttgggttgatgagtttgaggctatggatt

SEQ ID NO: 10 - DNA, 20 bp, Arabidopsis thaliana
ggctattgttggtggtggtc

SEQ ID NO: 11 - DNA, 20 bp, Arabidopsis thaliana
tccagcttcagaaacctgct

SEQ ID NO: 12 - DNA, 60 bp, Arabidopsis thaliana
gctattgttggtggtggtcctgctggtttagccgtggctcagcaggtttctgaagctgga

SEQ ID NO: 13 - DNA, 20 bp, Arabidopsis thaliana
caaactccactgtggtctgc

SEQ ID NO: 14 - DNA, 20 bp, Arabidopsis thaliana
aacccagtggcatcaagaac

SEQ ID NO: 15 - DNA, 69 bp, Arabidopsis thaliana
aaactccactgtggtctgcagtgacggtgtaaagattcaggcttccgtggttcttgatgccactgggtt

SEQ ID NO: 16 - DNA, 20 bp, Arabidopsis thaliana
tggtgttcatggattggaga

SEQ ID NO: 17 - DNA, 20 bp, Arabidopsis thaliana
gaacgttgggatcttgctgt

SEQ ID NO: 18 - DNA, 79 bp, Arabidopsis thaliana
tggtgttcatggattggagagacaaacatctggactcatatcctgagctgaagaacggaacagcaagatcccaacgttc

SEQ ID NO: 19 - DNA, 20 bp, Arabidopsis thaliana
ggggatcaatgtgaagagga

SEQ ID NO: 20 - DNA, 20 bp, Arabidopsis thaliana
ccacaacccgttgaggtaag

SEQ ID NO: 21 - DNA, 89 bp, Arabidopsis thaliana
ggggatcaatgtgaagaggattgaggaagacgagcgttgtgtgatcccgatgggcggtcctttaccagtcttacctcaacgggttgtgg

SEQ ID NO: 22 - DNA, 20 bp, Arabidopsis thaliana
gcacccattcaccgagtagt

SEQ ID NO: 23 - DNA, 20 bp, Arabidopsis thaliana
atgttcaacaggtggggaaa

SEQ ID NO: 24 - DNA, 50 bp, Arabidopsis thaliana
gcacccattcaccgagtagtcgaggagacttttccccacctgttgaacat

SEQ ID NO: 25 - DNA, 20 bp, Arabidopsis thaliana
cagtttttgctttgcgttca

SEQ ID NO: 26 - DNA, 20 bp, Arabidopsis thaliana
ctgggcggatttcatctaaa

SEQ ID NO: 27 - DNA, 60 bp, Arabidopsis thaliana
cagtttttgctttgcgttcatttattgaagcctgcaaagatttagatgaaatccgcccag

SEQ ID NO: 28 - DNA, 20 bp, Arabidopsis thaliana
tcaagtgccttctggttgaa

SEQ ID NO: 29 - DNA, 20 bp, Arabidopsis thaliana
agtatgccaagtgccaaagg

SEQ ID NO: 30 - DNA, 70 bp, Arabidopsis thaliana
tcaagtgccttctggttgaagtggttgcaaatgccttttactacaatacccctttggcacttggcatact

SEQ ID NO: 31 - DNA, 20 bp, Arabidopsis thaliana
tcgacactgacaacggtgat

SEQ ID NO: 32 - DNA, 20 bp, Arabidopsis thaliana
ggtactgatggcacggagac

SEQ ID NO: 33 - DNA, 80 bp, Arabidopsis thaliana
tcgacactgacaacggtgatgatgaaactgatgatgctggtgcattggctgcagtgggatgtctccgtgccatcagtacc

SEQ ID NO: 34 - DNA, 20 bp, Arabidopsis thaliana
cgagtctcgtcgatttcctc

SEQ ID NO: 35 - DNA, 20 bp, Arabidopsis thaliana
ttaaagcgaggctaggcaga

SEQ ID NO: 36 - DNA, 91 bp, Arabidopsis thaliana
cgagtctcgtcgatttcctccgggaggagacttgaaattcgtgactttccgattgtgaattccccgatggatctgcctagcctcgctttaa

SEQ ID NO: 37 - DNA, 20 bp, Arabidopsis thaliana
gtctccgtgccatcagtacc

SEQ ID NO: 38 - DNA, 20 bp, Arabidopsis thaliana
agcattttccgcattattgg

SEQ ID NO: 39 - DNA, 100 bp, Arabidopsis thaliana
gtctccgtgccatcagtaccattcttgaatctatcagtagtctccctcatctttatggtcagattgaaccacagttactgccaataatgcggaaaatgct

SEQ ID NO: 40 - DNA, 20 bp, Arabidopsis thaliana
tgtctctgacgacgaggttg

SEQ ID NO: 41 - DNA, 20 bp, Arabidopsis thaliana
cgtcctcttcagcgtcatct

SEQ ID NO: 42 - DNA, 50 bp, Arabidopsis thaliana
tgtctctgacgacgaggttgtccccgtagaagatgacgctgaagaggacg

SEQ ID NO: 43 - DNA, 20 bp, Arabidopsis thaliana
ggagaacgcaaacgtctgtt

SEQ ID NO: 44 - DNA, 20 bp, Arabidopsis thaliana
aagggtgattgcagcatttc

SEQ ID NO: 45 - DNA, 59 bp, Arabidopsis thaliana
ggagaacgcaaacgtctgttgaacatagcaatgcattgcggaaatgctgcaatcaccct

SEQ ID NO: 46 - DNA, 20 bp, Arabidopsis thaliana
aggaaccctcgattcgatct

SEQ ID NO: 47 - DNA, 20 bp, Arabidopsis thaliana
tcgaagctctagccatcgac

SEQ ID NO: 48 - DNA, 69 bp, Arabidopsis thaliana
aggaccctcgattcgatctctcagacgaaatcaggattcgtagaggcgcgtcgatggctagagcttcga

SEQ ID NO: 49 - DNA, 20 bp, Arabidopsis thaliana
ccctcgattcgatctctcag

SEQ ID NO: 50 - DNA, 19 bp, Arabidopsis thaliana
gaagaaacttcccgcttcg

SEQ ID NO: 51 - DNA, 79 bp, Arabidopsis thaliana
cctcgattcgatctctcagacgaaatcaggattcgtagaggcgcgtcgatggctagagctcgaagcgggaagtttcttc

SEQ ID NO: 52 - DNA, 20 bp, Arabidopsis thaliana
cagcaaacgtgagaaggcta

SEQ ID NO: 53 - DNA, 20 bp, Arabidopsis thaliana
tggaagcattttgggagtct

SEQ ID NO: 54 - DNA, 89 bp, Arabidopsis thaliana
cagcaaacgtgagaaggctagactcaaagaaatgcagaagatgaagaagcagaaaattcagcaaatcttagactcccaaaatgcttcca

SEQ ID NO: 55 - DNA, 19 bp, Arabidopsis thaliana
gccgattttgtcctgtcct

SEQ ID NO: 56 - DNA, 20 bp, Arabidopsis thaliana
atgtcgaatttccctgcaac

SEQ ID NO: 57 - DNA, 100 bp, Arabidopsis thaliana
gccgattttgtcctgtcctgcgtgctgtgaaatttctcggtaatcccgaggaaagaagacatattcgtgaagaactgctagttgcagggaaattcgacat

SEQ ID NO: 58 - DNA, 19 bp, Arabidopsis thaliana
agcacagagcctcgccttt

SEQ ID NO: 59 - DNA, 23 bp, Arabidopsis thaliana
ggtgtgcacttttattcaactgg

SEQ ID NO: 60 - DNA, 20 bp, Arabidopsis thaliana
agagaagtggggtggctttt

SEQ ID NO: 61 - DNA, 20 bp, Arabidopsis thaliana
agggcagtgatctccttctg

SEQ ID NO: 62 - DNA, 20 bp, Arabidopsis thaliana
agaggcgtacagggatagca